(19) United States
(12) Patent Application Publication
(10) Pub. No.: US 2016/0097063 A1
(43) Pub. Date: Apr. 7, 2016

(54) Production Of Enantiopure alpha-Hydroxy Carboxylic Acids From Alkenes By Cascade Biocatalysis

(71) Applicant: NATIONAL UNIVERSITY OF SINGAPORE, Singapore (SG)

(72) Inventors: Zhi Li, SG (SG); Shuke Wu, SG (SG)

(73) Assignee: National University of Singapore, SG (SG)

(21) Appl. No.: 14/892,824

(22) PCT Filed: May 22, 2014
(86) PCT No.: PCT/SG2014/000221

§ 371 Date: Nov. 20, 2015

Related U.S. Application Data
(60) Provisional application No. 61/826,165, filed on May 22, 2013.

(51) Int. Cl. C12P 7/42; C12N 9/14; C12P 7/22; C12N 9/02

(52) U.S. Cl. CPC C12P 7/42; C12N 9/0071; C12N 9/14; C12P 7/22

(57) ABSTRACT

The invention provides compositions comprising an alkene epoxidase and a selective epoxide hydrolase, such as a recombinant microorganism comprising a first heterologous nucleic acid encoding an alkene epoxidase and a second heterologous nucleic acid encoding a selective epoxide hydrolase. Exemplary alkene epoxidases include StyAB, while exemplary selective epoxide hydrolases include epoxide hydrolases from Sphingomonas, Solanum tuberosum, or Aspergillus. The invention also provides non-toxic methods of making enantiomerically pure vicinal diols or enantiomerically pure alpha-hydroxy carboxylic acids using these compositions and microorganisms.

### RELATED APPLICATION

This application claims the benefit of U.S. Provisional Application No. 61/826,165, filed on May 22, 2013. The entire teachings of the above application are incorporated herein by reference.

### BACKGROUND OF THE INVENTION

Enantiomerically pure α-hydroxy carboxylic acids are an important class of fine chemicals with broad application in many industries. Traditional methods to manufacture these optically active compounds involve the use of very toxic and hazardous prussia acid, HCN. Accordingly, a need exists for methods of making enantiomerically pure α-hydroxy carboxylic acids (or vicinal diols) that do not rely on toxic materials such as HCN.

### SUMMARY OF THE INVENTION

The invention provides, inter alia, green biocatalysis methods (HCN free) to prepare α-hydroxy carboxylic acids (or vicinal diols) from cheap and readily available terminal alkenes, as well as compositions, recombinant microorganisms, and nucleic acids useful in these methods. The synthetic route involves selective epoxidation, hydrolysis and oxidation steps, and all of them can be performed in mild conditions and in an economic way. The whole reactions take place in a cascade manner in one pot (without the isolation and purification of intermediates) by using cells, isolated enzymes, immobilized enzymes, immobilized cells or a mixture of these cells and enzymes. Examples of the appropriate catalysts are engineered recombinant whole cells expressing multiple enzymes or recombinant enzyme catalysts. The concept was proven by the successful production of (S)-mandelic acid from styrene in two approaches: (1) multiple cells strategy: engineering three recombinant E. coli cells expressing styrene monooxygenase, epoxide hydrolase, alcohol dehydrogenase and aldehyde dehydrogenase, respectively, and using the mixed cells for one-pot reactions; (2) single cell strategy: engineering one recombinant E. coli cell coexpressing these enzymes and performing the cascade reactions in one cell. The model synthetic methodology can be extended to other alkene substrates to produce other chiral α-hydroxy carboxylic acids in high enantiomeric excess (ee) and high yield.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a plasmid map of pRSFduet-StyAB*SpEH. LacI: Lac repressor for controlling the gene expression; RSF ori: plasmid replicate origin; Kn: kanamycin resistance gene; StyA: first component of SMO (styrene monooxygenase); StyB: second component of SMO; SpEH: epoxide hydrolase from Sphingomonas sp. HXN-200.

FIG. 2 is a micrograph of an SDS gel of cell proteins of three different E. coli recombinants co-expressing SMO and SpEH. Lane 1: Marker (Invitrogen see blue plus two); Lanes 2 & 3: E. coli (P-StyA-P-StyB*SpEH); Lanes 4 & 5: E. coli (P-StyA*StyB-P-SpEH); Lanes 6 & 7: E. coli (P-StyA*StyB*SpEH).

FIG. 3 provides a bar graph of production of (S)-phenylethane-1,2-diol from styrene by using whole cells of three different E. coli recombinants expressing SMO and SpEH (StyA*B*SpEH; StyA-P-StyB*SpEH; StyA*B—P-SpEH). (S)-Phenylethane-1,2-diol; Sty: styrene. For each data series, from left to right, the values are for: (S)-diol for 1 hour, (S)-diol for 3 hours, (S)-diol for 5 hours, styrene for 5 hours.

FIG. 4 is a graph of a chiral HPLC chromatogram of bioproduct (S)-phenylethane-1,2-diol from cascade biotransformation of styrene with E. coli (pRSFduet-StyAB*SpEH). S-Diol: (S)-phenylethane-1,2-diol; R-Diol: (R)-phenylethane-1,2-diol.

FIG. 5 is a plasmid map of pRSFduet-StyAB*StEH. LacI: Lac repressor for controlling the gene expression; RSF ori: plasmid replicate origin; Kn: kanamycin resistance gene; StyA: first component of SMO (styrene monooxygenase); StyB: second component of SMO; StEH: epoxide hydrolase from Solanum tuberosum.

FIG. 6 is a micrograph of an SDS gel of cell proteins of three different E. coli recombinants co-expressing SMO and StEH. Lane 1: Marker (Invitrogen see blue plus two); Lanes 2 & 3: E. coli (P-StyA-P-StyB*StEH); Lanes 4&5: E. coli (P-StyA*StyB-P-StEH); Lane 6: E. coli (P-StyA*StyB*StEH).

FIG. 7 provides a bar graph of production of (R)-phenylethane-1,2-diol from styrene by using whole cells of three different E. coli recombinants expressing SMO and StEH (StyA*B*StEH; StyA*B-P-StEH; StyA-P-StyB*StEH). (R)-Diol: (R)-Phenylethane-1,2-diol; Sty: styrene. For each data series, from left to right, the values are for: (R)-diol for 1 hour, (R)-diol for 3 hours, (R)-diol for 5 hours, styrene for 5 hours.

FIG. 8 is a graph of a chiral HPLC chromatogram of bioproduct (R)-phenylethane-1,2-diol from cascade biotransformation of styrene with E. coli (pRSFduet-StyAB*StEH). S-Diol: (S)-phenylethane-1,2-diol; R-Diol: (R)-phenylethane-1,2-diol.

FIG. 9 is a plot of concentration over time, illustrating oxidation of racemic phenylethane-1,2-diol with resting cells of E. coli (Sp1184, a new cloned ADH from Sphingomonas) and E. coli (AlkH). Reaction conditions: 20 mM substrate and 5 g cdw/L each recombinant cell.

FIG. 10 is a plot of a reverse phase HPLC chromatogram of bioproduct mandelic acid from phenylethane-1,2-diol using wild type acetic acid bacterium Gluconobacter oxydans 621H. Diol: phenylethane-1,2-diol; Man: mandelic acid; IS: Internal Standard (1 mM benzyl alcohol).

FIG. 11 is a plot of a reverse phase HPLC chromatogram of bioproduct (5)-mandelic acid from cascade biotransformation of styrene using mixed cells of E. coli (pRSFduet-StyAB*SpEH), E. coli (pET28a-AlkJ) and E. coli (pET28a-AlkH). Diol: (S)-Phenylethane-1,2-diol; Internal Standard: 1 mM benzyl alcohol.

FIG. 12 is a cartoon of plasmid constructs provided by the invention. Genetic construction of upstream module: StyAB*SpEH on four different plasmids: pACYC, pCDF, pETduet, and pRSF for co-expression of SMO and SpEH. Downstream module: AlkJ*EcALDH on four different plasmids: pACYC, pCDF, pETduet, and pRSF for co-expression of AlkJ (from Pseudomonas putida) and EcALDH (from Escherichia coli).

FIG. 13 provides bar graphs of production of (S)-mandelic acid (S-MA) from 100 mM styrene by 12 different recombinant E. coli strains that contained different combinations of plasmids of upstream module and downstream module. The values represent the S-MA yield at 20 hours, and are the average results of three independent experiments.

FIG. 14 is a graph of concentration over time in the production of (S)-mandelic acid (S-MA) from 120 mM styrene (STY) by the best E. coli strain (ACRS5) under optimized conditions in small scale. The values represent the average results of three independent experiments.

### DETAILED DESCRIPTION OF THE INVENTION

A description of example embodiments of the invention follows.

In a first aspect, the invention provides compositions containing an alkene epoxidase and a selective epoxide hydrolase. These compositions can be in a variety of forms, including, for example:
a) a recombinant microorganism expressing the alkene epoxidase and selective epoxide hydrolase;
b) a protein extract of the microorganism of a);
c) purified alkene epoxidase and purified selective epoxide hydrolase;
d) purified alkene epoxidase and purified selective epoxide hydrolase, wherein the purified enzymes are attached to solid supports.
e) a composition of any one of a)-d), further comprising a diol oxidation system; or
f) any combination of the foregoing.

A “recombinant microorganism” is a product of man that is markedly different from a microorganism (e.g., bacteria, unicellular fungus, protist, et cetera) that exists in nature. In particular embodiments provided by the invention, the recombinant microorganism is markedly different from a microorganism that exists in nature due to the presence of a heterologous nucleic acid, which may be maintained on an exogenous plasmid or stably maintained in the genome of the microorganism. “Heterologous” refers to materials that are not associated in nature. In some embodiments, for example, a heterologous nucleic acid construct includes a nucleic acid (or plurality of nucleic acids) associated with a nucleic acid from another species, but, in other embodiments, can include a recombinant construct where two nucleic acids from the same species are associated together in a non-naturally-occurring way, such as associating different promoters and coding sequences.

An “alkene epoxidase” is an enzyme capable of catalyzing the epoxidation of an alkene. In particular embodiments, the alkene epoxidase is capable of the epoxidation of a terminal alkene, such as an aryl terminal alkene. In some embodiments, the alkene epoxidase is enantioselective. In some embodiments, the alkene epoxidase is not enantioselective. Exemplary alkene epoxidases include monooxygenases (such as styrene monooxygenases (see, e.g., SEQ ID NOs: 1, 2), P450 monooxygenases (see, e.g., SEQ ID NOs: 3, 4), alkene monooxygenases), lipases (e.g., that are capable of lipase-mediated oxidation), and peroxidases. In some embodiments, the alkene epoxidase is a variant of any of the foregoing, e.g., the enzyme is a styrene monooxygenase, such as StyAB, or an alkene epoxidase at least 60% identical to StyAB.

An “selective epoxide hydrolase” is an enzyme that may be regioselective or enantioselective when hydrolysing an epoxide to a vicinal diol. In some embodiments, a selective epoxide hydrolase produces an abundance of one enantiomer, or, if applicable, diastereomer, (at least 55, 60, 65, 70, 75, 80, 85, 90, 91, 92, 93, 94, 95, 96, 97, 98, 99%, or more, of total enantiomers (ee) or diastereomers (de)) when hydrolysing an epoxide to a vicinal diol. In some embodiments, the selective epoxide hydrolase is regioselective. In certain embodiments, the selective epoxide hydrolase is enantioselective. Exemplary selective epoxide hydrolases include epoxide hydrolases from Sphingomonas (see, e.g., SEQ ID NO: 5), Solanum tuberosum (see, e.g., SEQ ID NO: 6), and Aspergillus (see, e.g., SEQ ID NO: 7). In some embodiments, the selective epoxide hydrolase produces an excess of an S enantiomer of a vicinal diol. In other embodiments, the selective epoxide hydrolase produces an excess of an R enantiomer of a vicinal diol.

Suitable solid supports for use in the invention include: 1) inorganic carriers such as SiO₂, porous glass or ion-oxides; 2) natural organic carriers such as polysaccharides (Agarose), crosslinked dextrans (Sepharose) or cellolose; 3) synthetic organic carriers such as acrylamide derivatives (co-polymers), acrylate-derivatives (co-polymers), vinylacetate derivatives (co-polymers), polyamides, polystyrene derivatives, polypropylenes or polymer-coated ion oxide particles.

In related aspects, the invention provides recombinant microorganisms that contain a first heterologous nucleic acid encoding an alkene epoxidase and a second heterologous nucleic acid encoding a selective epoxide hydrolase. These enzymes can be selected as already described, above, and includes variants as described, infra.

In some embodiments, the recombinant microorganism also includes a nucleic acid encoding a diol oxidation system. In particular embodiments, the nucleic acid encoding a diol oxidation system is a heterologous nucleic acid.

A “diol oxidation system” comprises one or more enzymes that catalyze the oxidation of a diol to an aldehyde or, in more particular embodiments, a carboxylic acid. In some embodiments, the diol oxidation system is an alcohol oxidation system from an acetic acid bacterium, such as Gluconobacter (see, e.g., SEQ ID NO: 11). In some embodiments, the diol oxidation system comprises an alcohol dehydrogenase (such as AlkJ from Pseudomonas (see, e.g., SEQ ID NO: 8), horse liver alcohol dehydrogenase (see, e.g., SEQ ID NO: 10), or alcohol dehydrogenase from Sphingomonas (see, e.g., SEQ ID NO: 9)) or a dihydrodiol dehydrogenase, or a variant thereof that is at least 60% homologous or identical at the amino acid level to the reference sequence. In particular embodiments, the alcohol oxidation system comprises an aldehyde dehydrogenase, such as AlkH from Pseudomonas (see, e.g., SEQ ID NO: 12), aldehyde dehydrogenase from Escherichia (see, e.g., SEQ ID NO: 13), aldehyde dehydrogenase from Sphingomonas (see, e.g., SEQ ID NOs: 14, 15) or a variant thereof that is at least 60% homologous or identical at the amino acid level to the reference sequence. In certain embodiments, the diol oxidation system comprises an alcohol dehydrogenase together with an aldehyde dehydrogenase or a dihydrodiol dehydrogenase together with an aldehyde dehydrogenase. In these embodiments, the aldehyde dehydrogenase and dihydrodiol dehydrogenase can be contained in a single nucleic acid construct or in two or more nucleic acid constructs that are co-transformed or exist in separate organisms that are cocultured. In particular embodiments, the alcohol oxidation system comprises an alcohol oxidase, such as AldO from Streptomyces (see, e.g., SEQ ID NO 16).

In some embodiments, an enzyme useful in the present invention is a sequence variant of any of the exemplary enzymes described herein (e.g., alkene epoxidase, selective epoxide hydrolase, or diol oxidation system (alcohol dehydrogenase, aldehyde dehydrogenase, or both); the exemplary sequences described herein are “reference sequences”) which retain at least about: 30, 40, 50, 60, 70, 80, 90, 95, or 100% of the reference enzymatic activity-“variant enzyme(s).” In some embodiments, variant enzymes are at least about: 60, 65, 70, 75, 80, 85, 90, 95, 96, 97, 98, 99%, or more, homologous or identical at the amino acid level to a reference amino acid sequence described above or a functional fragment thereof—e.g., over a length of about: 50, 55, 60, 65, 70, 75, 80, 85, 90, 95, or 100% of the length of the mature reference sequence. In certain embodiments, a nucleic acid encoding a variant enzyme hybridizes to a nucleic acid encoding one of the reference sequences under highly stringent hybridization conditions. “Highly stringent hybridization” conditions means at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. Where a variant enzyme bears a strong structural and functional relation to a reference sequence (as defined by a percentage of homology or identity or hybridization under highly stringent hybridization conditions), amino acid variations will take into account regions of the protein that are important for its function, such as conserved domains defined for the reference sequences or as identified by sequence alignments to available homologous sequences from other organisms. Amino acid substitutions can be conservative or non-conservative (as defined by PAM30, PAM50, PAM100, PAM150 or BLOSUM62). The skilled artisan will appreciate that amino acid variations in conserved regions should generally be conservative, while non-conservative amino acid variations outside of conserved regions are better tolerated.

In some embodiments, the recombinant microorganism is a bacterium, such as E. coli.

In a related aspect, the invention provides compositions containing the recombinant microorganism provided by the invention.

In some embodiments, the compositions provided by the invention include a second recombinant microorganism comprising a nucleic acid encoding a diol oxidation system. In more particular embodiments, the numerical ratio of the first recombinant microorganism and second recombinant microorganism produces a relative maximum of yield of enantiomerically pure alpha-hydroxy carboxylic acid from an alkene.

“Enantiomerically pure” means one enantiomer or diastereomer represents at least about: 80, 85, 90, 91, 92, 93, 94, 95, 96, 97, 98, 99%, or more, of total enantiomers or diastereomers.

In some embodiments, a composition provided by the invention is a liquid, such as a two phase liquid with an aqueous phase and a second phase with improved solubility for an alkene, relative to the aqueous phase.

In certain embodiments, a composition provided by the invention includes an alkene suitable for conversion to a diol or alpha carboxylic acid by the composition.

In another aspect, the invention provides methods of non-toxic production of an enantiomerically pure vicinal diol. These methods entail contacting a suitable composition provided by the invention or suitable microorganism provided by the invention with an alkene in a solution under conditions where the recombinant microorganism expresses the alkene epoxidase and selective epoxide hydrolase, thereby producing the enantiomerically pure vicinal diol. In these embodiments, the vicinal diol is preferably produced from the alkene without intervening purification steps. In particular embodiments, the alkene is a terminal alkene, an aryl alkene, or an aryl terminal alkene. In more particular embodiments, the alkene is any one of the substrates shown in any one of Tables 2-8 and Schemes 1-5, or a salt or ester thereof. These methods can be used to generate, inter alia, any one of the products shown in any one of Tables 2-8 and Schemes 1-5, or a salt or ester thereof

“Non-toxic production,” e.g., of an enantiomerically pure vicinal diol or alpha-hydroxy carboxylic acid, means the production does not require prussic acid (HCN) or its derivatives.

In a related aspect, the invention provides methods of non-toxic production of an enantiomerically pure alpha-hydroxy carboxylic acid. These methods include the steps of contacting suitable compositions provided by the invention or suitable recombinant microorganisms provided by the invention with a terminal alkene in a solution under conditions where the recombinant microorganism expresses the alkene epoxidase and selective epoxide hydrolase and the diol oxidation system is expressed, thereby producing the enantiomerically pure alpha-hydroxy carboxylic acid. In particular embodiments, the alpha-hydroxy carboxylic acid is produced from the terminal alkene without intervening purification steps. In certain embodiments, the terminal alkene is any one of the substrates shown in any one of Tables 2 and 3 and Schemes 1 and 2, or a salt or ester thereof. These methods can be used to generate any one of the products shown in any one of Tables 2 and 3 and Schemes 1 and 2, or a salt or ester thereof. In particular embodiments, the product is Mandelic acid, or a salt or ester thereof.

The methods provided by the invention enable high yield production of vicinal diols or alpha-hydroxy carboxylic acids, such as yields of at least about: 30, 40, 50, 60, 65, 70, 75, 80, 85, 90, 91, 92, 93, 94, 95, 96, 97, 98, 99%, or more. The methods provided by the invention also provide high ee or de vicinal diols or alpha-hydroxy carboxylic acids, such as at least at least about: 30, 40, 50, 60, 65, 70, 75, 80, 85, 90, 91, 92, 93, 94, 95, 96, 97, 98, 99%, or more, ee or de. In more particular embodiments, the methods provided by the invention provide both high yield and high ee or de of vicinal diols or alpha-hydroxy carboxylic acids.

In some embodiments, the methods provided by the invention are performed in a two phase liquid comprising an aqueous phase and a second phase with improved solubility for an alkene relative to the aqueous phase.

Various reaction conditions, such as buffers, pH, temperature, et cetera, can be used consonant with the invention. In some embodiments, the conditions are selected to achieve maximal yield and/or ee or de. For example, in some embodiments, the methods provided by the invention are performed in an solution with buffers, such as phosphate buffer, citrate buffer, Tris buffer and HEPES buffer. In some embodiments, the methods provided by the invention are performed in an aqueous system with pH of about 3 to about 12, in more particular embodiments, with pH of from about 6 to about 9. In some embodiments, the methods provided by the invention are performed at a temperature of about 0° C. to about 90° C., such as from about 20° C. to about 40° C. Any combination of these conditions can be used in the methods provided by the invention.

In other aspects, the invention provides nucleic acids encoding constructs described in the exemplification, including variants thereof, e.g., with different backbones (origins of replication, selectable markers, et cetera), varied promoters, or variant enzymes as described herein. The invention also provides methods of making any of the products described in the tables, schemes and exemplifications in the application.

### EXEMPLIFICATION

Enantiomerically pure α-hydroxy carboxylic acids are an important class of fine chemicals which have broad application in chemical, pharmaceutical and cosmetics industries. For example, (R)-mandelic acid is a versatile intermediate for the synthesis of several pharmaceuticals (e.g., β-lactam antibiotics) as well as a useful resolving agent in chiral separation processes, with a production scale of at least several hundred tons per year at the price of USD60 per kg. (S)-mandelic acid is also very useful and is applied in some chiral resolution processes. Optically pure chloro- and fluoro-substituted mandelic acid derivatives are essential for some pharmaceutical syntheses. (R)-2-hydroxy-4-phenyl butyric acid is the key chiral precursor to manufacture a group of angiotensin-converting enzyme (ACE) inhibitors (such as enalapril, lisinopril, and ramipril). Optically active (R)-2-hydroxybutyric acid is an important building block for the production of biodegradable material for biomedical, pharmaceutical and environmental applications. This class of chiral compounds is so important that intensive effort has been made to develop the methods to produce them.

The production of these enantiomerically pure α-hydroxy carboxylic acids can be achieved by chemical methods through metal-based catalysts. However, they suffer from the costly and harmful nature of metal-based catalysts. Thus, most of these optical pure α-hydroxy carboxylic acids are produced by enzymes or microbes in industry currently. There are two industry applied biosynthetic strategies to chiral α-hydroxy carboxylic acids. (1) Enzymatic hydrolysis of cyano groups of racemic cyanohydrins, synthesized chemically by adding prussic acid, HCN, to the aldehydes. (2) Enantioselective biocatalytic hydrocyanation of aldehydes, followed by chemically converting the product chiral cyanohydrins to chiral α-hydroxy carboxylic acids. Although the current biocatalysis systems fulfill the industrial requirements of enantio purity and yield, there is an unavoidable safety and environmental issue in both processes: they require the use of highly toxic and dangerous prussia acid, HCN, or its salt form (such as KCN) as a key reactant. The highly toxic HCN and its salts not only create serious hazards to the process, people and environment, but also increase the production cost because of the special instruments and extreme care necessary for handling these very toxic compounds. There are also some other synthetic routes reported in literature, such as kinetic resolution of racemic ester form of α-hydroxy carboxylic acids and enantioselective reduction of α-keto acids or esters; however, these methods have the drawbacks of low yield (kinetic resolution) and limited availability of substrates (kinetic resolution and selective reduction). These weaknesses largely hinder the commercialization of these processes. Therefore, novel, green, and efficient methods are urgently needed to produce chiral α-hydroxy carboxylic acids from readily available and cheap starting substrates.

In this invention, we describe a novel cascade biocatalysis route to produce enantiomerically pure α-hydroxy carboxylic acids from the readily available and cheap terminal alkenes through epoxidation, hydrolysis and oxidations.

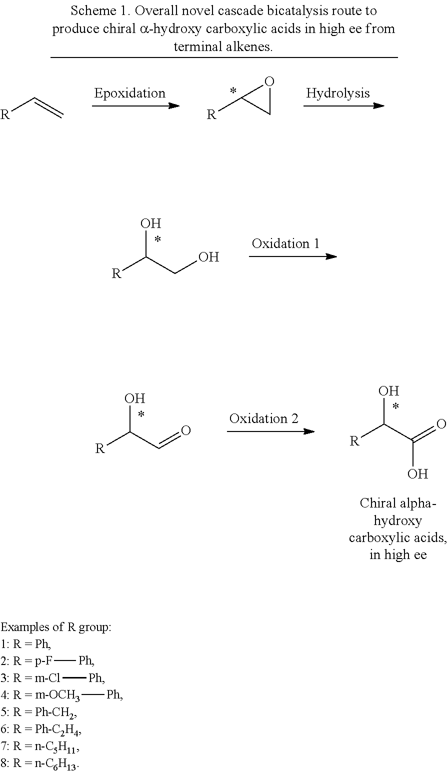

Many enzymes and microorganisms are discovered or engineered that are useful for these reactions.

TABLE 1
Inventory of enzymes used in isolation or whole cells for the four
reactions in the cascade biocatalysis route.^{[a]}

| Epoxidation | Hydrolysis | Oxidation 1 | Oxidation 2 |
|---|---|---|---|
| Styrene | Epoxide hydrolase from | Alcohol dehydrogenase | Aldehyde dehydrogenase |
| monooxygenase | Sphingomonas | AlkJ from Pseudomonas | AlkH from Pseudomonas |
| P450 monooxygenase | Epoxide hydrolase from | Alcohol dehydrogenase | Aldehyde dehydrogenase |
| | Solanum tuberosum | from horse liver | from Escherichia |
| Alkene | Epoxide hydrolase from | Alcohol dehydrogenase | Aldehyde dehydrogenase |
| monooxygenase | Aspergillus | from Sphingomonas | from Sphingomonas |
| Lipase-mediated | | Dihydrodiol | Alcohol dehydrogenase |
| oxidation | | dehydrogenase | |

| | |
|---|---|
| Peroxidase | Alcohol oxidation system from acetic |
| | acid bacterium, e.g. Gluconobacter |

^{[a]}The enzymes are used in isolation or whole cells, and they are combined in different forms and ratios for one-pot cascade biocatalysis.

For instance, the terminal alkenes are catalyzed by monooxygenase (such as styrene monooxygenase, P450 monooxygenase, and alkene monooxygenase, etc.), peroxidase, or lipase-mediated oxidation to produce chiral epoxides; these epoxides are then selectively hydrolyzed by epoxide hydrolase (e.g., epoxide hydrolases from Sphingomonas sp. HXN-200, Solanum tuberosum and Aspergillus niger) to form vicinal diols; in the next step, some alcohol dehydrogenases (e.g., alkJ from Pseudomonas putida, horse liver alcohol dehydrogenase, and dihydrodiol dehydrogenase, etc.) or alcohol oxidase are applied to perform terminal oxidation of these vicinal diols to α-hydroxy aldehydes; lastly, the α-hydroxy aldehydes are then oxidized to the enantiomerically pure α-hydroxy carboxylic acids with an oxidation enzyme, such as aldehyde dehydrogenase or alcohol dehydrogenase. In some embodiments, cascade biocatalysis is performed in one pot, allowing for green, efficient, and economical production of enantiomerically pure α-hydroxy carboxylic acids. Biocatalysis has similar reaction conditions, and thus cascade biocatalysis could be carried out in one pot to provide a new and simple method for chemical synthesis. In comparison with multi-step synthesis, which is often used in the production of pharmaceuticals and fine chemicals, one-pot cascade reactions could avoid the expensive and energy-consuming isolation and purification of intermediates, minimize waste generation, and overcome the possible thermodynamic hurdles in multi-step synthesis. Owing to fast development of modern biotechnology, multiple enzymes can be co-expressed inside one cell while the whole cell serves as a powerful catalyst for a serial of cascade reactions in one pot. Alternatively, these enzymes can be separately expressed in several cells, purified individually, or immobilized, and the biocatalyst (enzymes, cells, immobilized enzymes, and immobilized cells) can be mixed together in one pot to carry out the reaction.

The substrate terminal alkenes are readily and cheaply available from the petrochemical industry (by hydrocarbon cracking). Important examples of terminal alkenes are aromatic and aliphatic terminal alkenes. For instance, styrene is a prototype aromatic terminal alkene produced in a very large commodity at very low price. Styrene and substituted styrenes are model and also very useful substrates for this invention. Aliphatic terminal alkenes such as 1-hexene and 1-heptene, and aromatic alkenes such as 1-pentanene allylbenzene and 4-phenyl-1-butene, are also good substrates in this invention to prepare the corresponding chiral α-hydroxy carboxylic acids in high enantiomeric excess (ee).

The whole cells of the recombinant E. coli containing the necessary enzymes for desired reaction steps are a suitable biocatalyst for the cascade reactions. In this case, all the chemical reactions take place inside a single cell. To construct the recombinant biocatalyst, the enzymes are cloned and expressed heterogeneously in E. coli cells. The multiple enzymes can be put in one plasmid as an artificial operon (which facilitates the co-expression of all the enzymes) or separately in different, but compatible, plasmids. After transforming the plasmids into the E. coli strain, the multiple enzymes are co-expressed and the whole recombinant cells serve as a good biocatalyst for the cascade reactions.

In a representative example of producing chiral (substituted) mandelic acid from (substituted) styrene,

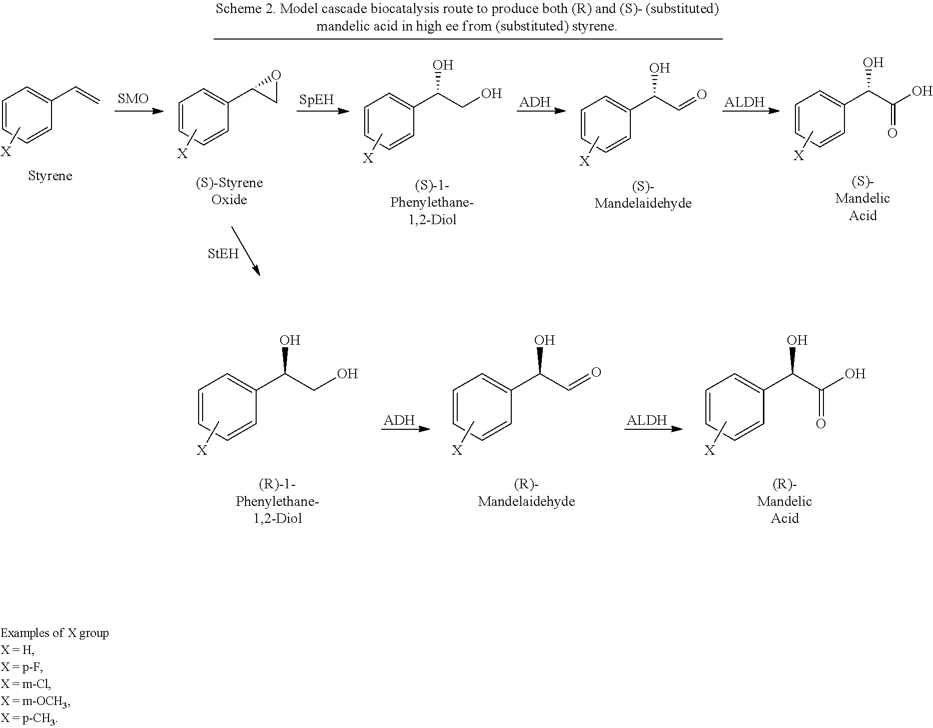

styrene monooxygenase (SMO, a two component (StyA & StyB) flavin-dependent monoxygenase) from styrene degradation strain Pseudomonas sp. VLB120 was used as the first enzyme to produce (S)-styrene oxide from styrene. The two components StyA and StyB were cloned from the template plasmid pSPZ10 to the plasmid pRSFduet and formed an artificial operon for easy expressing. The successful construction pRSFduet-StyAB allowed for co-expressing the two components of SMO in the host E. coli with very high SMO activity for the enantioselective epoxidation of styrene to (S)-styrene oxide. In the second step, two complementary selective epoxide hydrolases (EH) were used to produce (S)-phenylethane-1,2-diol and (R)-phenylethane-1,2-diol, respectively. EH from Sphingomonas sp. HXN-200 (SpEH) transformed (S)-styrene oxide to (S)-diol. The SpEH was cloned from Sphingomonas sp. HXN-200 and then combined with SMO on the same plasmid in three different expression cassettes. The engineered E. coli recombinants expressed the SMO and SpEH very well and efficiently converted styrene to (S)-1-phenylethane-1,2-diol. To produce (R)-diol, the gene of enantioselective EH from Solanum tuberosum was commercially synthesized and cloned into the same SMO-expressing plasmids. Similarly, three different expression cassettes combining SMO and StEH were constructed. All of the resulting E. coli strains expressed SMO and StEH very well and could efficiently convert styrene to (R)-1-phenylethane-1,2-diol. For the final two step reactions in the cascade biocatalysis route to produce chiral (substituted) mandelic acid from (substituted) styrene (Scheme 2), alkJ, a terminal alcohol dehydogenase from the alkane degradation strain Pseudomonas putida, was utilized to oxidize the diol to aldehyde, and alkJ or alkH (aldehyde dehydogenase from the same Pseudomonas putida strain) was used for the subsequent oxidation of the aldehyde to α-hydroxy acid. The alkJ and alkH were cloned from OCT plasmid to pET28a plasmid. The constructions pET28a-alkJ and pCDF-StyAB*SpEH (which is subcloned from plasmid pRSF-StyAB*SpEH) were co-transformed into E. coli host strains. These recombinant E. coli co-expressing SMO, SpEH and AlkJ were able to produce (S)-mandelic acid from styrene.

In another important alternative strategy in cascade biocatalysis, cells of multiple recombinant or wild type stains expressing the necessary enzymes were combined for one-pot biocatalysis. These recombinant E. coli cells individually expressed one or two enzymes for one or two step reactions. When several whole cells were mixed together, they catalyzed the total cascade reactions to produce enantiomerically pure α-hydroxy carboxylic acids in one pot. By using this strategy, the ratio of different enzymes can be easily adjusted and optimized by changing the ratio of cells of different recombinants to maximize the production of final product. In these embodiments, the cascade biocatalysis can be better than that with one strain co-expressing the multiple enzymes.

In a representative example of producing chiral (substituted) mandelic acid in high ee from (substituted) styrene (Scheme 2) via the multiple cell strategy, the aldehyde dehydogenase (alkH) from Pseudomonas putida strain was cloned from OCT plasmids to pET28a plasmid resulting in pET28a-alkH. Three different recombinant E. coli cells containing recombinant plasmids, pRSFduet-StyAB*SpEH, pET28a-alkJ and pET28a-alkH, were grown, and the necessary enzymes were expressed separately. These cell were mixed together to perform the cascade reactions from styrene to (S)-mandelic acid with high conversion and high yield.

In the case of transforming other terminal alkenes to enantiomerically pure α-hydroxy carboxylic acids, similar synthetic pathways can be achieved by using the recombinant E. coli co-expressing these similar enzymes or cells of multiple recombinants expressing these enzymes. For instance, from 1-hexene to enantiopure 2-hydroxyhexanoic acid, the following enzymes can be used: (1) P450pyr from Sphingomonas sp. HXN-200 for the epoxidation of 1-hexene to 1-hexene oxide; (2) EH from Sphingomonas sp. HXN-200 for the hydrolysis to form 1,2-hexene diol; (3) horse liver alcohol dehydrogenase for the oxidization of the 1,2-hexene diol to 2-hydroxyhexanoic acid. In this invention, we have shown that the triple mutant of P450pyr (P450pyrTM) catalyzes the conversion of 1-hexene to 1-hexene oxide in high ee. The P450 monooxygenase system could be engineered to produce other enantiopure terminal epoxides.

Because of the nature of enzymes and biocatalysis, the cascade bioreactions are better performed in aqueous phase. For low-concentration biotransformation, an aqueous one phase system fulfills the requirement and can achieve the final product easily. However, the substrates, alkenes, are generally quite hydrophobic and can be harmful for the cell and enzyme. Thus, an organic:aqueous biphase reaction system is a better choice for high-concentration biotransformation. The alkenes and intermediate epoxides have better solubility in organic phase, while the diols, acids, cells and enzymes are mostly in the aqueous phase. By applying the biphase reaction system, the problems of low solubility and inhibition of substrates are solved. In addition, the product α-hydroxy carboxylic acids are easily separated from the unreacted substrates and some intermediates (epoxides).

Other forms of biocatalyst that also could be applied to synthesize α-hydroxy carboxylic acids in high ee are encompassed by the invention. These include isolated enzymes, enzymes immobilized on nano or micro size support (such as magnetic nano particles) to increase their stability and reusability, wild type microbial cells, and recombinant cells immobilized on some carriers. By utilizing isolated enzymes, immobilized enzymes, or immobilized cells, the cascade biocatalysis can be performed to produce α-hydroxy carboxylic acids in high ee with good yield. A mixture of different forms of biocatalyst is also a suitable system to carry out the cascade biocatalysis.

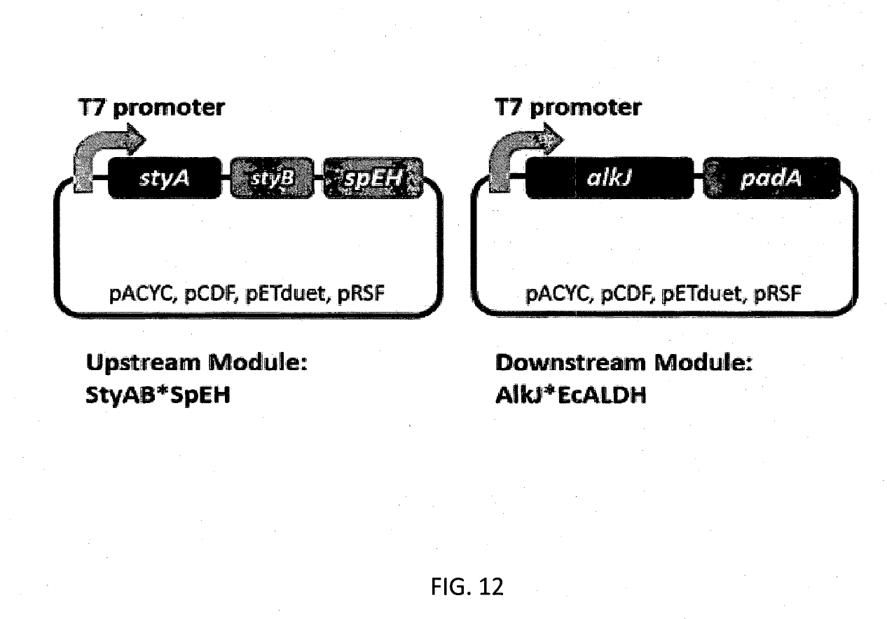

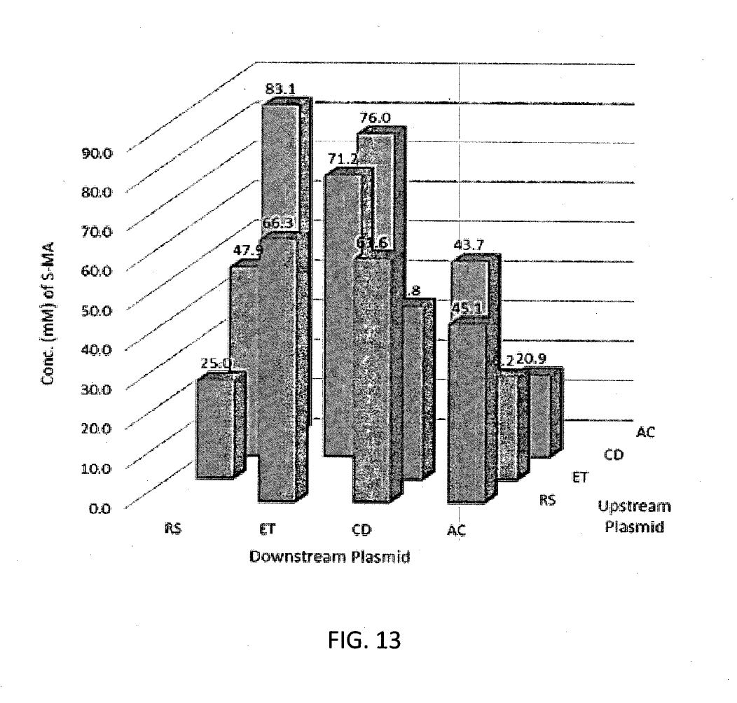

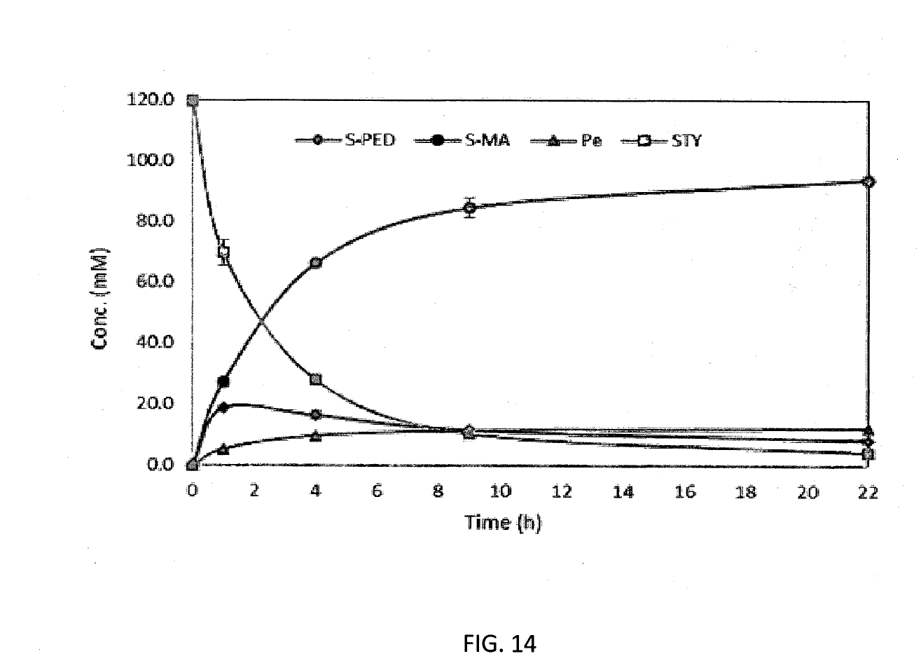

In a representative example of producing chiral (S)-mandelic acid from styrene (Scheme 2), a modular optimization of multiple enzymes was employed to develop a very efficient recombinant E. coli strain. The SMO and SpEH were cloned to an artificial operon (StyAB*SpEH) as upstream module and sub-cloned to four different plasmids: pACYC, pCDF, pETduet, pRSF (FIG. 12). These four plasmids have different antibiotic resistant genes and different copy numbers in living E. coli; thus, the SMO and SpEH were expressed at different levels. On the other hand, the other two enzymes, AlkJ and EcALDH, were also cloned to one artificial operon (AlkJ*EcALDH) as downstream module and sub-cloned to the four plasmids. The combination of these upstream modules and downstream modules led to 12 different recombinant E. coli strains, where four enzymes (SMO, SpEH, AlkJ, and EcALDH) were expressed at different levels. The 12 strains were further tested for converting 100 mM styrene in small scale. The results showed significantly different performance of these strains (FIG. 13). The best one, E. coli (ACRS5), containing upstream module on pACYC plasmid and downstream module on pRSF plasmid, could efficiently produce about 83±7 mM (S)-mandelic acid from 100 mM styrene in 20 hours. Further optimization of the reaction system led to production of 94±2 mM (about 14.3 g/L) (S)-mandelic acid from 120 mM styrene in 22 hours (FIG. 14). These results demonstrate that one recombinant whole cell expressing multiple enzymes in optimal levels is a good catalyst to perform cascade catalysis. The modular optimization method could be used to optimize the enzyme expression in vivo.

In representative examples of producing substituted (S)-mandelic acids from substituted styrenes (Scheme 2), the best strain, E. coli (ACRS5), was used to transform various different substituted styrenes, such as 2-fluorostyrene, 3-fluorostyrene, 4-fluorostyrene, 3-chlorostyrene, 4-chlorostyrene, and 3-methylstyrene. The reaction was performed in aqueous-n-hexadecane two phase system with 20 mM substrate and 10 g cdw/L resting cells of E. coli (ACRS5) as catalysts. The reactions were quite efficient in that all six substituted styrenes were converted 78-98%, and the yield of the final product substituted (S)-mandelic acids was also high (72%-98%). The accumulation of diol intermediates or by-products was either insignificant (<10%) or not observed (<1%). More importantly, the enantiomeric excess of three of the substituted (S)-mandelic acids was determined to be a very high 96.6-98.4%. This proves the cascade biocatalytic process is consistently and highly enantioselective. The production of different substituted (S)-mandelic acids also demonstrated the relatively broad substrate scope of the reaction cascade. It is one of the elegant examples of cascade biocatalysis for enantiopure chemical production.

### Example 1

### Genetic Engineering of E. coli Recombinant Expressing SMO and SpEH

The first enzyme, styrene monooxygenase (SMO), catalyzed the epoxidation of styrene to (S)-styrene oxide. The enzyme SMO was comprised of two components (polypeptides): StyA and StyB. In order to optimize the activity of SMO, these two components were expressed together in two ways: (1) two promoters respectively drove the expression of StyA and StyB, and the construction is P-StyA-P-StyB; (2) there was only one promoter and StyA and StyB were expressed as one operon (P-StyAB). In the construction of P-StyA-P-StyB, StyA was first cloned using the template pSPZ10 and the following primers: A CTG TCA TGA AAA AGC GTATCG GTA TTG TTG G (SEQ ID NO: 17) and A CTG GAA TTC TCA TGC TGC GAT AGT TGG TGC GAA CTG (SEQ ID NO: 18) to pRSFduet plasmid (available from Novagen) at NcoI and EcoRI restriction site to produce pRSFduet-StyA plasmid; and then StyB component was cloned by the primers A CTG CAT ATG ACG CTG AAA AAA GAT ATG GC (SEQ ID NO: 19) and A CTG GGT ACC TCA ATT CAG TGG CAA CGG GTT GC (SEQ ID NO: 20) to the intermediate plasmid pRSFduet-StyA by NdeI and KpnI restriction site to produce P-StyA-P-StyB. In the construction of P-StyAB, StyB component was cloned by the primers A CTG GAA TTC TAA GGA GAT TTC AAA TGA CGC TGA AAA AAG ATA TGG C (SEQ ID NO: 21) and A CTG GGT ACC TCA ATT CAG TGG CAA CGG GTT GC (SEQ ID NO: 20) to the same intermediate plasmid pRSFduet-StyA by EcoRI and KpnI restriction site. The two different constructions P-StyA-P-StyB and P-StyAB both co-expressed the two components of SMO in the host E. coli (T7 expression strain from NEB or BL21DE3 strain from Novagen). The recombinant E. coli strains containing the plasmid P-StyA-P-StyB or P-StyAB were grown in 1 mL LB medium containing 50 mg/L kanamycin at 37° C. and then 2% inoculated into 25 mL TB medium (50 mg/L kanamycin), and, when OD₆₀₀ reached 0.6, 0.5 mM IPTG was added to induce the expression of protein. The cells continued to grow and express protein for 5 hours at 30° C. before they were harvested by centrifuge. The cells were resuspended in 5 mL 100 mM KPB buffer (pH=8.0) and OD₆₀₀ was measured. The cells were employed as a catalyst to transform styrene to (S)-styrene oxide in an aqueous buffer:hexadecane two phase system (2 mL:2 mL) with 2% glucose for cofactor regeneration. The cell loading was 10 g cdw/L and the substrate styrene loading was 100 mM (10.4 g/L). The concentration of styrene and styrene oxide were measured by GC during the reaction. In a very short time (3 hours), more than 90% of styrene was converted to (S)-styrene oxide using E. coli (P-StyAB). The enantiomeric excess (ee) of styrene oxide was determined to be >99% by chiral HPLC (Daicel AS-H column, Hex:IPA=90:10, 0.5 mL per min). These results prove the success of construction of P-StyA-P-StyB and P-StyAB and the high activity of recombinant E. coli cells containing them.

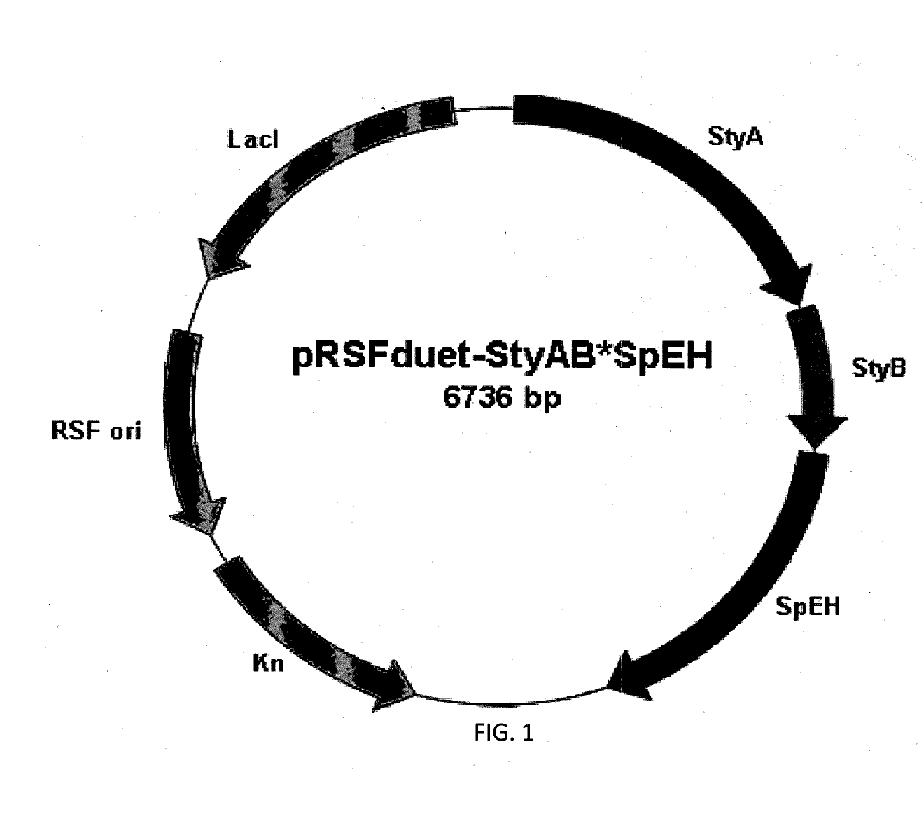

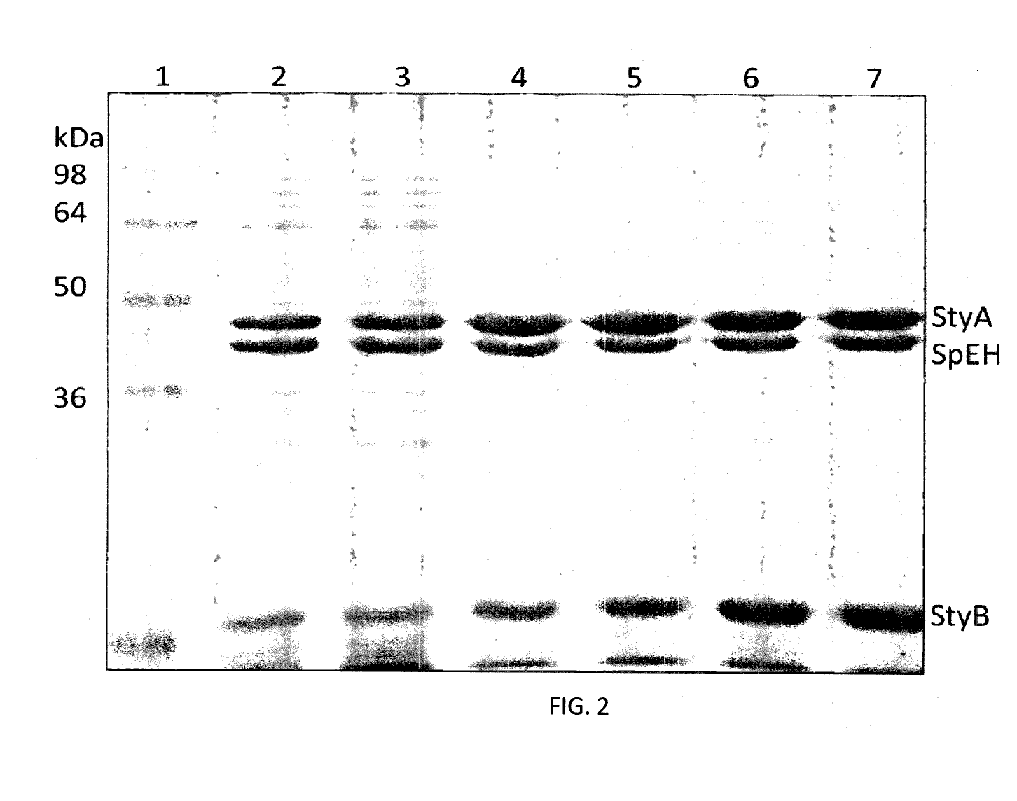

The EH from Sphingomonas sp. HXN-200 (SpEH) was chosen to transform (5)-styrene oxide to produce (S)-diol. The SpEH was first cloned from the genome of HXN-200 to pRSFduet plasmid (from Novagen, NdeI and XhoI restriction sites) by the following primers: A TCG CAT ATG ATG AAC GTC GAA CAT ATC CGC CC (SEQ ID NO: 22) and A TCG CTC GAG TCA AAG ATC CAT CTG TGC AAA GGC C (SEQ ID NO: 23). It was combined with SMO by subcloning into two SMO expressing plasmids: P-StyA-P-StyB and P-StyAB. Three different expression cassettes were tried that were different in the number and position of the promoters (P represents T7 promoter and * represents the direct connection of two genes with RBS but without promoter): P-StyA-P-StyB*SpEH, P-StyA*StyB-P-SpEH and P-StyA*StyB*SpEH. To construct P-StyA-P-StyB*SpEH, SpEH was cloned to P-StyA-P-StyB by KpnI and XhoI restriction site using the primers A CTG GGT ACC TAA GGA GAT ATA TCA TGA TGA ACG TCG AAC ATA TCC GCC C (SEQ ID NO: 24) and A TCG CTC GAG TCA AAG ATC CAT CTG TGC AAA GGC C (SEQ ID NO: 23). To construct P-StyA*StyB-P-SpEH, SpEH was cloned to P-StyAB by NdeI and XhoI restriction site using the primers A TCG CAT ATG ATG AAC GTC GAA CAT ATC CGC CC (SEQ ID NO: 22) and A TCG CTC GAG TCA AAG ATC CAT CTG TGC AAA GGC C (SEQ ID NO: 23). To construct P-StyA*StyB*SpEH, SpEH was cloned to P-StyAB by KpnI and XhoI restriction site using the primers A CTG GGT ACC TAA GGA GAT ATA TC A TGA TGA ACG TCG AAC ATA TCC GCC C (SEQ ID NO: 24) and A TCG CTC GAG TCA AAG ATC CAT CTG TGC AAA GGC C (SEQ ID NO: 23). All of them expressed the enzymes well (12% SDS gel; see FIG. 2) and converted styrene to (S)-1-phenylethane-1,2-diol. The best of these three constructions was P-StyA*StyB*SpEH (pRSF-StyAB*SpEH; see the plasmid map in FIG. 1), in which SMO and SpEH were co-expressed under the control of one promoter.

### Example 2

### Production of (S)-Phenylethane-1,2-Diol from Styrene Via Cascade Biocatalysis Using E. coli Cells Expressing SMO and SpEH

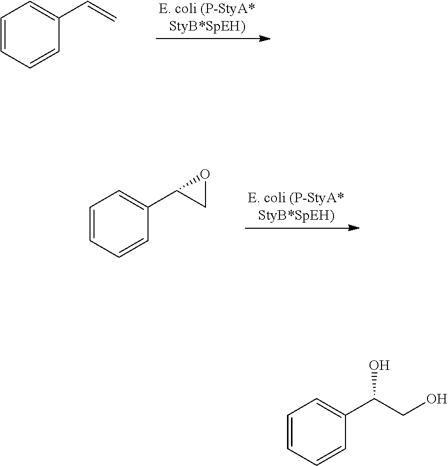

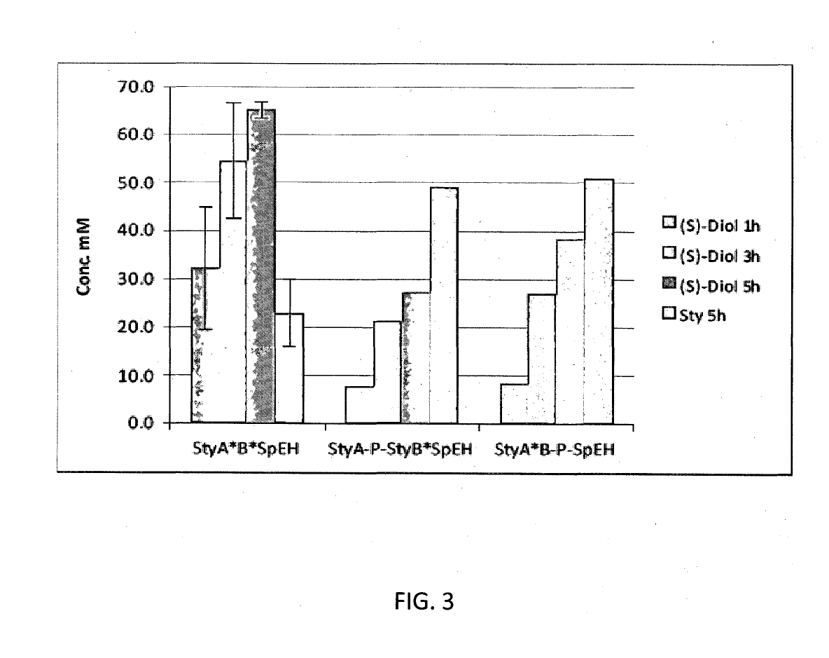

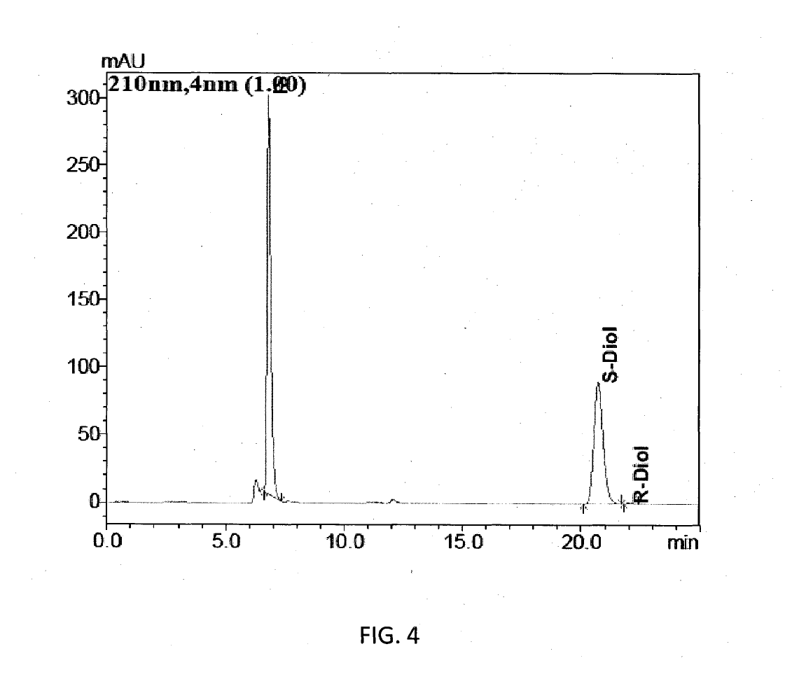

Three recombinant E. coli strains (T7 expression strain from NEB or BL21DE3 strain from Novagen) containing the plasmid P-StyA-P-StyB*SpEH, P-StyA*StyB-P-SpEH or P-StyA*StyB*SpEH were grown in 1 mL LB medium containing 50 mg/L kanamycin at 37° C. and then 2% inoculated into 25 mL TB medium (50 mg/L kanamycin). When OD₆₀₀ reached 0.6, 0.5 mM IPTG was added to induce the expression of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL 2 mL) for biotransformation of 100 mM styrene (2% glucose for cofactor regeneration). The reaction was conducted at 30° C. and 300 rpm in a 100-mL flask for 5 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. All three recombinant cells co-expressing SMO and SpEH produced (S)-phenylethane-1,2-diol from styrene, and the best result was about 65 mM (9.0 g/L) (S)-phenylethane-1,2-diol obtained at 5 hours with the recombinant E. coli P-StyA*StyB*SpEH (pRSF-styAB*SpEH) (FIG. 3). The enantiomeric excess (ee) of the product (S)-phenylethane-1,2-diol was determined to be >99% by chiral HPLC (Daicel AS-H column, Hex:IPA=90:10, 0.5 mL/min) (FIG. 4). These results show that our constructed recombinant strains are powerful catalysts for the cascade biotransformation of styrene to (S)-phenylethane-1,2-diol.

### Example 3

### Production of Substituted (S)-Phenylethane-1,2-Diols from Substituted Styrenes Via Cascade Biocatalysis Using E. coli Cells Expressing SMO and SpEH

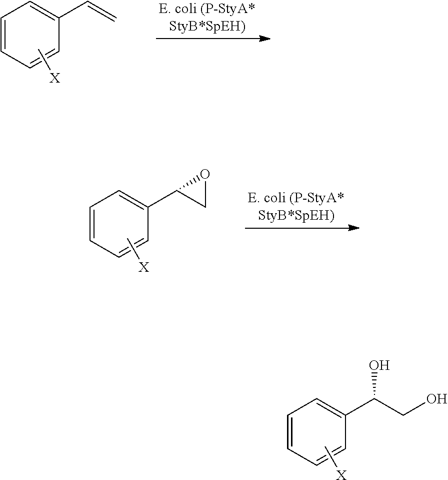

In addition to non-substituted mandelic acid, many chiral substituted mandelic acids are also useful intermediates. To fully explore the potential for other substituted (S)-mandelic acids production, we first tested the existing system to produce the key intermediates, substituted (S)-phenylethane-1,2-diols. The E. coli (P-StyA*StyB*SpEH) was grown in 1 mL LB medium containing 50 mg/L kanamycin at 37° C. and then 2% inoculated into 25 mL M9-Glu-Y medium (standard M9 medium plus 20 g/L glucose and 5 g/L yeast extract) with 50 mg/L kanamycin. When OD₆₀₀ reached 0.6, 0.5 mM IPTG was added to induce the expressing of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL:2 mL) for biotransformation of 20 mM different substituted styrenes.

TABLE 2
Conversion of styrene derivatives to (S)-diols by E. coli (P-StyA*StyB*SpEH)^{[a]}

| | Activity | Conversion | | Yield | ee (%) |
|---|---|---|---|---|---|
| Substrate | (U/g cdw)^{[b]} | (5)^{[c]} | Product | (%)^{[c]} | (configuration)^{[d]} |
| 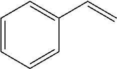 | 46 | >99 | 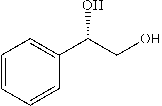 | 92 | 98.1(S) |
| 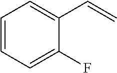 | 11 | 94 | 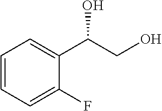 [SMILES: OC[C@@H](O)c1ccccc1F] | 94 | 98.6(S) |
| 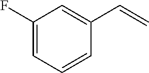 | 41 | >99 | 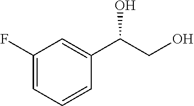 [SMILES: OC[C@@H](O)c1cccc(F)c1] | >99 | 98.4(S) |
| 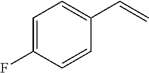 | 33 | >99 | 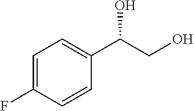 [SMILES: OC[C@@H](O)c1ccc(F)cc1] | 88 | 97.9(S) |
| 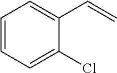 | 4 | 31 | 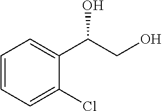 [SMILES: OC[C@@H](O)c1ccccc1Cl] | 34 | 92.2(S) |
| 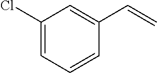 | 22 | 96 | 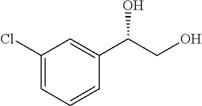 [SMILES: OC[C@@H](O)c1cccc(Cl)c1] | >99 | 97.5(S) |
| 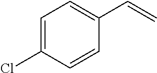 | 20 | 67 | 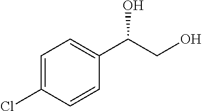 [SMILES: OC[C@@H](O)c1ccc(Cl)cc1] | 73 | 97.8(S) |
| 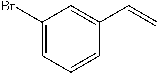 | 8 | 80 | 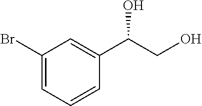 [SMILES: OC[C@@H](O)c1cccc(Br)c1] | 67 | 97.5(S) |
| 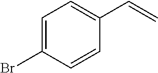 | 7 | 40 | 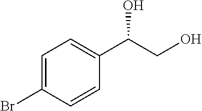 [SMILES: OC[C@@H](O)c1ccc(Br)cc1] | 34 | 97.7(S) |
| 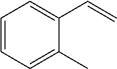 | 5 | 36 | 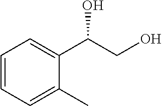 | 34 | 65.7(S) |
| 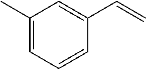 | 11 | 97 | 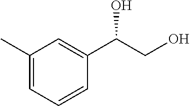 | 91 | 93.1(S) |
| 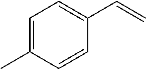 | 12 | 98 | 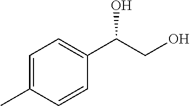 | 86 | 93.9(S) |
| 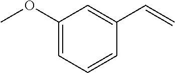 | 55 | >99 | 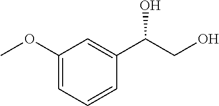 | 96 | 97.6(S) |
| 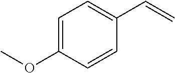 | 26 | >99 | 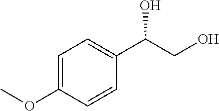 | 67 | 83.2(S) |
| 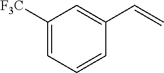 | 2 | 41 | 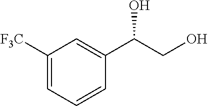 [SMILES: OC[C@@H](O)c1cccc(C(F)(F)F)c1] | 46 | 97.6(S) |
| 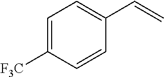 | 2 | 31 | 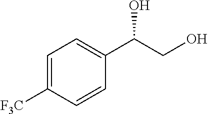 [SMILES: OC[C@@H](O)c1ccc(C(F)(F)F)cc1] | 25 | 97.5(S) |

^{[a]}The reaction was performed in a two-phase system consisting of KPB buffer (200 mM, pH 8.0, containing 2% glucose and 10 g cdw/L cells) and n-hexadecane (1:1) with 20 mM substrate for 8 hours.
^{[b]}Activity was determined at initial 30 min.
^{[c]}Conversion and yield were determined by HPLC analysis.
^{[d]}ee value was determined by chiral HPLC analysis.

The reaction was conducted at 30° C. and 300 rpm in a 100-mL flask for 8 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. The ee of the product diols was determined by chiral HPLC. As listed in Table 2, most of the (S)-diols can be produced in high ee (14 out of 16 achieved >90% ee) from substituted styrenes by E. coli (P-StyA*StyB*SpEH) cells. Worth noting is that the yields of many produced (S)-diols are also good (>80%). These results demonstrate the broad substrate scope of our constructed recombinant biocatalyst E. coli (P-StyA*StyB*SpEH) and its great application potential in tandem biocatalysis to produce substituted (S)-mandelic acids.

### Example 4

### Genetic Engineering of Recombinant E. coli Co-Expressing SMO and StEH

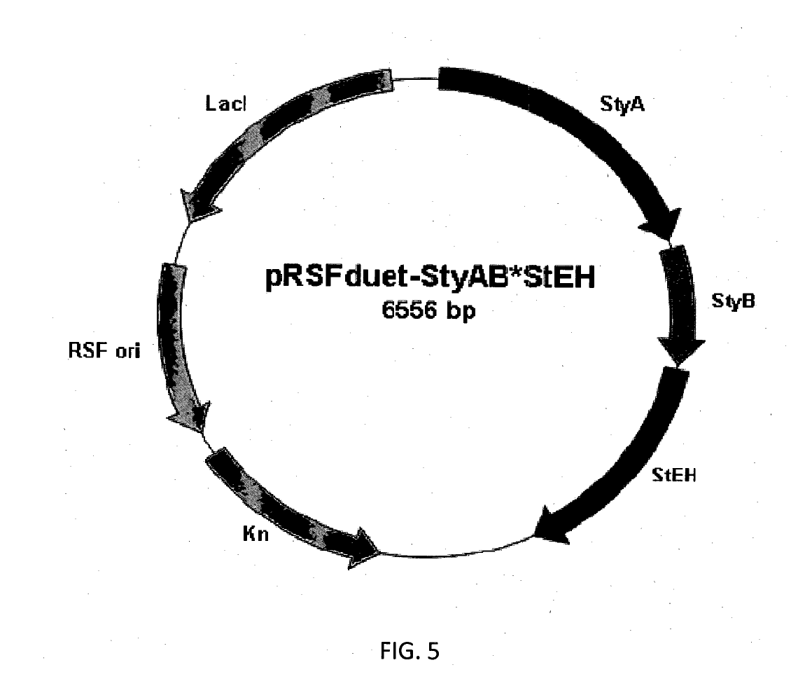

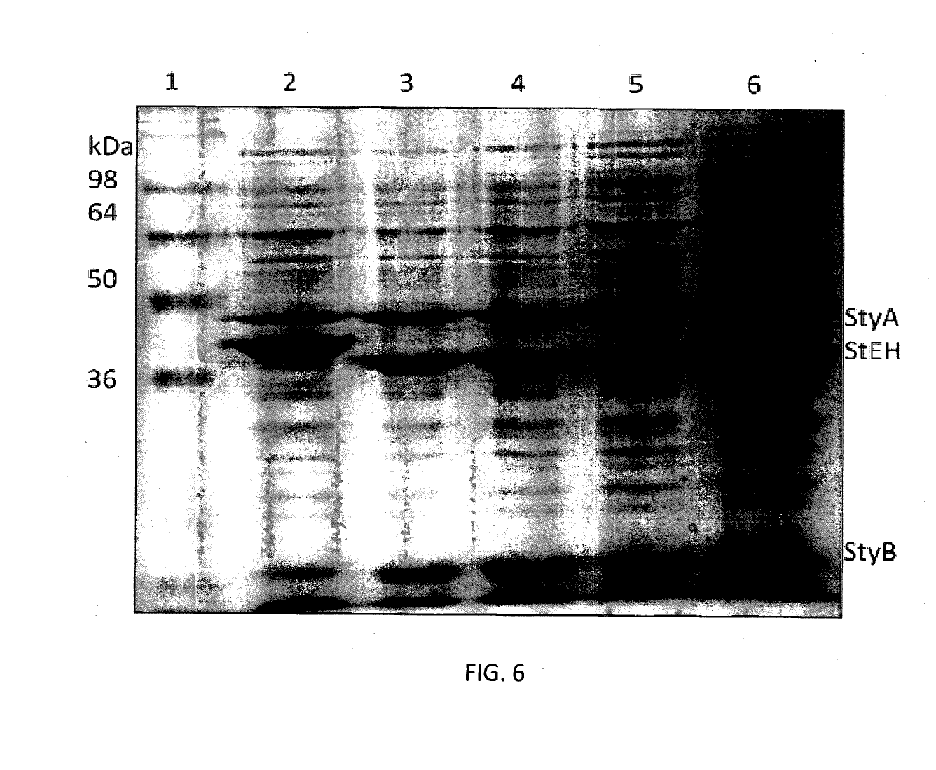

StEH from Solanum tuberosum catalyzed the hydrolysis of (S)-styrene oxide to (R)-1-phenylethane-1,2-diol. The commercially synthesized StEH gene (by Genscript) was cloned into the pRSFduet plasmids (from Novagen) by NdeI and XhoI restriction site using the primers A CTG CAT ATG GAG AAA ATC GAA CAC AAG ATG (SEQ ID NO: 25) and A CTG CTC GAG TTA GAA TTT TTG AAT AAA ATC (SEQ ID NO: 26). After the success of this cloning, StEH was subcloned to two SMO expressing plasmids (P-StyA-P-StyB and P-StyAB). To explore the different expression pattern of these enzymes, three expression cassettes combining SMO and StEH were constructed: P-StyA-P-StyB*StEH, P-StyA*StyB-P-StEH and P-StyA*StyB*StEH. To construct P-StyA-P-StyB*StEH, StEH was cloned to P-StyA-P-StyB by KpnI and XhoI restriction site using the primers A CTG GGT ACC TAA GGA GAT ATA TCA TGG AGA AAA TCG AAC ACA AGA T (SEQ ID NO: 27) and A CTG CTC GAG TTA GAA TTT TTG AAT AAA ATC (SEQ ID NO: 26). To construct P-StyA*StyB-P-StEH, StEH was cloned to P-StyAB by NdeI and XhoI restriction site using the primers A CTG CAT ATG GAG AAA ATC GAA CAC AAG ATG (SEQ ID NO: 25) and A CTG CTC GAG TTA GAA TTT TTG AAT AAA ATC (SEQ ID NO: 26). To construct P-StyA*StyB*StEH, StEH was cloned to P-StyAB by KpnI and XhoI restriction site using the primers A CTG GGT ACC TAA GGA GAT ATA TCA TGG AGA AAA TCG AAC ACA AGA T (SEQ ID NO: 27) and A CTG CTC GAG TTA GAA TTT TTG AAT AAA ATC (SEQ ID NO: 26). All of these recombinant E. coli expressed the enzymes very well (12% SDS gel; see FIG. 6) and catalyzed the conversion of styrene to (R)-1-phenylethane-1,2-diol. The best of these three expression cassettes was P-StyA*StyB*StEH (pRSF-StyAB*StEH, constructed by pRSF-StyAB with StEH using KpnI and XhoI restriction sites, FIG. 5), in which SMO and StEH were co-expressed under the control of one promoter.

### Example 5

### Production of (R)-Phenylethane-1,2-Diol from Styrene by SMO and StEH Co-Expressing Whole Cells

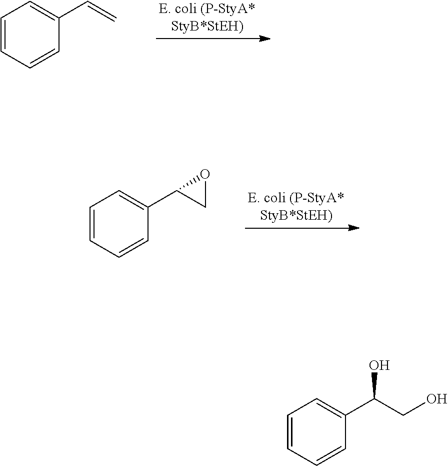

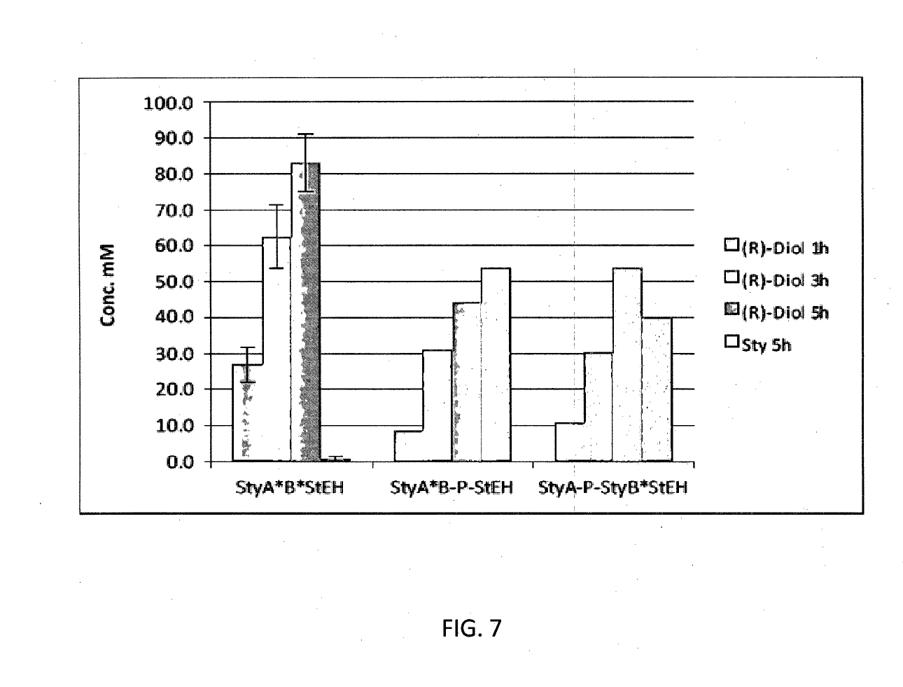

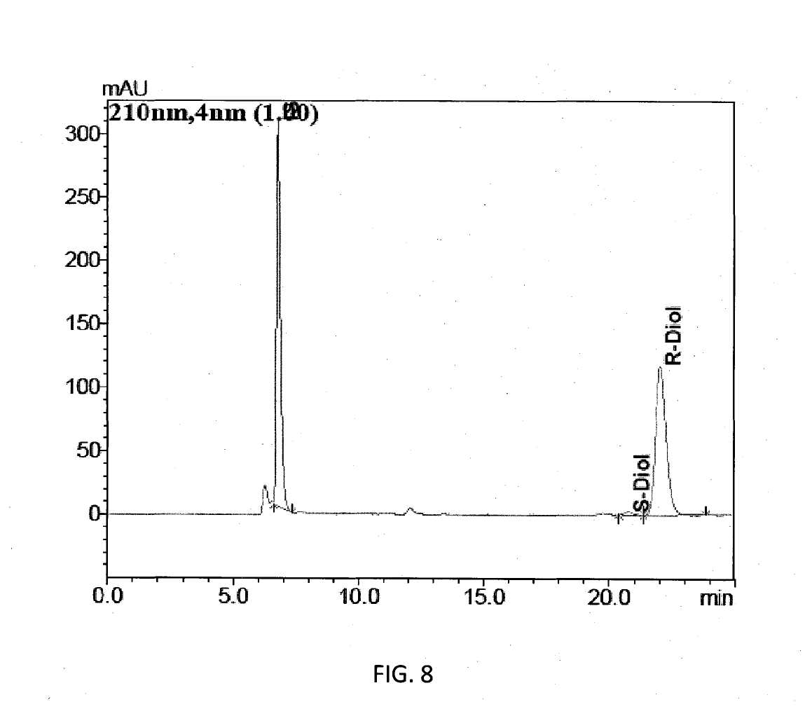

Three recombinant E. coli strains (T7 expression strain from NEB or BL21DE3 strain from Novagen) containing the plasmid P-StyA-P-StyB*StEH, P-StyA*StyB-P-StEH or P-StyA*StyB*StEH were grown in 1 mL LB medium containing 50 mg/L kanamycin at 37° C. and then 2% inoculated into 25 mL TB medium (50 mg/L kanamycin). When OD₆₀₀ reached 0.6, 0.5 mM IPTG was added to induce the expression of enzymes. The cells continued to grow for 12 hours at 22° C. before they were harvested by centrifuge (5000 g, 5 mins). The cells were resuspended in 100 mM KPB buffer (pH=8.0) to 10 g cdw/L and then mixed with hexadecane to produce an aqueous:organic two-phase system (2 mL:2 mL). 100 mM (10.4 g/L) styrene was added, plus 2% glucose for cofactor regeneration. The reaction was conducted at 30° C. and 300 rpm in a 100-mL flask for 5 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. In five hours, these three different recombinant cells co-expressing SMO and StEH produced (R)-phenylethane-1,2-diol from styrene, and the best result was about 90 mM (12.4 g/L) (R)-phenylethane-1,2-diol, obtained with E. coli StyA*StyB*StEH (pRSF-styAB*StEH) (FIG. 7). The ee of the product (R)-phenylethane-1,2-diol was determined to be 96% by chiral HPLC (Daicel AS-H column, Hex:IPA=90:10, 0.5 mL/min) (FIG. 8). These results show that our constructed recombinant cells are powerful catalysts for the cascade transformation of styrene to (R)-phenylethane-1,2-diol.

### Example 6

### Production of Substituted (R)-Phenylethane-1,2-Diols from Substituted Styrenes Via Cascade Biocatalysis Using E. coli Cells Expressing SMO and StEH

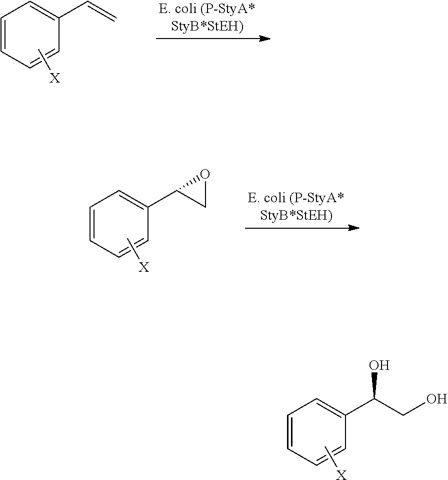

To research the potential for production of another enantiomer, substituted (R)-mandelic acid, we then tested another existing system to produce the key intermediates, substituted (R)-phenylethane-1,2-diols. The E. coli (P-StyA*StyB*StEH) was grown in 1 mL LB medium containing 50 mg/L kanamycin at 37° C. and then 2% inoculated into 25 mL M9-Glu-Y medium with 50 mg/L kanamycin. When OD₆₀₀ reached 0.6, 0.5 mM IPTG was added to induce the expressing of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL:2 mL) for biotransformation of 20 mM different substituted styrenes.

TABLE 3
Conversion of styrene derivatives to (R)-diols by E. coli (P-StyA*StyB*StEH)^{[a]}

| | Activity | Conversion | | Yield | ee (%) |
|---|---|---|---|---|---|
| Substrate | (U/g cdw)^{[b]} | (%)^{[c]} | Product | (%)^{[c]} | (configuration)^{[d]} |
| 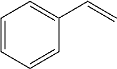 | 39 | >99 | 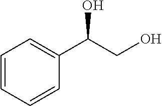 | 93 | 95.5(R) |
| 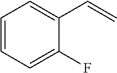 | 17 | 98 | 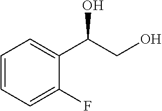 [SMILES: OC[C@H](O)c1ccccc1F] | 89 | 68.1(R) |
| 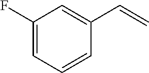 | 43 | >99 | 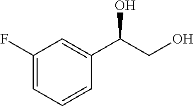 [SMILES: OC[C@H](O)c1cccc(F)c1] | >99 | 94.2(R) |
| 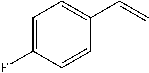 | 41 | >99 | 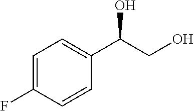 [SMILES: OC[C@H](O)c1ccc(F)cc1] | 90 | 95.2(R) |
| 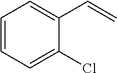 | 2 | 26 | 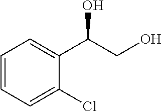 [SMILES: OC[C@H](O)c1ccccc1Cl] | 10 | 36.9(R) |
| 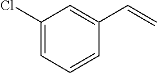 | 15 | 97 | 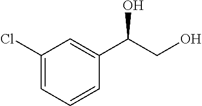 [SMILES: OC[C@H](O)c1cccc(Cl)c1] | >99 | 95.8(R) |
| 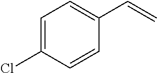 | 22 | 90 | 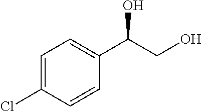 [SMILES: OC[C@H](O)c1ccc(Cl)cc1] | 97 | 95.6(R) |
| 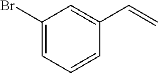 | 6 | 98 | 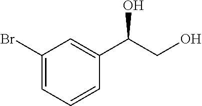 [SMILES: OC[C@H](O)c1cccc(Br)c1] | 86 | 84.2(R) |
| 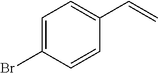 | 7 | 92 | 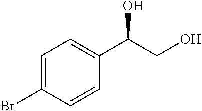 [SMILES: OC[C@H](O)c1ccc(Br)cc1] | 86 | 94.4(R) |
| 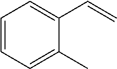 | 5 | 34 | 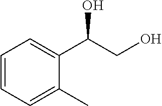 | 15 | 89.9(R) |
| 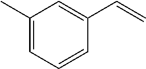 | 9 | 98 | 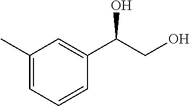 | 92 | 98.2(R) |
| 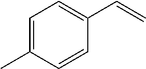 | 15 | >99 | 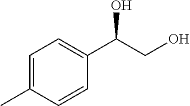 | 85 | 87.7(R) |
| 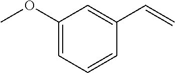 | 40 | >99 | 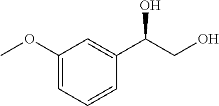 | >99 | 87.3(R) |
| 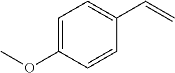 | 20 | >99 | 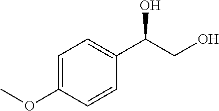 | 65 | 85.4(R) |
| 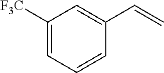 | 3 | 22 | 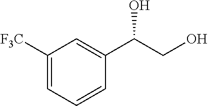 [SMILES: OC[C@@H](O)c1cccc(C(F)(F)F)c1] | 13 | 74.0(S) |
| 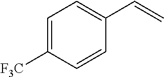 | 2 | 18 | 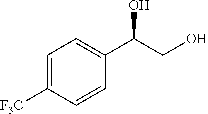 [SMILES: OC[C@H](O)c1ccc(C(F)(F)F)cc1] | 19 | 87.7(R) |

^{[a]}The reaction was performed in a two-phase system consisting of KPB buffer (200 mM, pH 8.0, containing 2% glucose and 10 g cdw/L cells) and n-hexadecane (1:1) with 20 mM substrate for 8 hours.
^{[b]}Activity was determined at initial 30 min.
^{[c]}Conversion and yield were determined by HPLC analysis.
^{[e]}ee value was determined by chiral HPLC analysis.

The reaction was conducted at 30° C. and 300 rpm in a 100-mL flask for 8 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. The ee of the product diols was determined by chiral HPLC. As can be seen in Table 2, many of the (R)-diols can be produced in high ee (12 out of 16 achieved >85% ee) with good yields (>80%) from substituted styrenes by E. coli (P-StyA*StyB*StEH) cells. The recombinant biocatalyst E. coli (P-StyA*StyB*StEH) was proven to accept various substituted styrenes and yield (R)-diols, which are subjected to tandem biocatalytic oxidation to produce substituted (R)-mandelic acids.

### Example 7

### Production of Mandelic Acid from Phenylethane-1,2-Diol Via Cascade Biocatalysis Using E. coli Cells Expressing Sp1814 and E. coli Cells Expressing AlkH

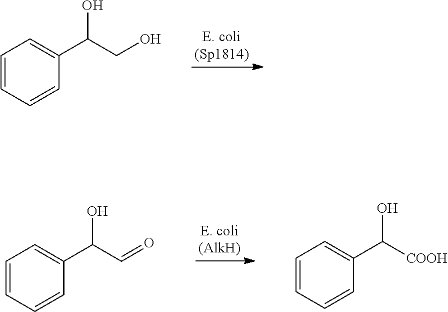 [SMILES: OCC(O)c1ccccc1 ; O=CC(O)c1ccccc1 ; O=C(O)C(O)c1ccccc1]

To produce α-hydroxy carboxylic acids, alcohol dehydrogenase was used to oxidize the diol intermediates. In addition to the alkJ from the alkane degradation strain Pseudomonas putida (Example 9), we found another alcohol dehydrogenase Sp1814 to be a promising catalyst. The Sp1814 was screened out from the many alcohol dehydrogenases from Sphingomonas sp. HXN-200. By using primers A CTG TCA TGA CGC AAG AGT CAG ATA ATA GTA CTT (SEQ ID NO: 28) and A CTG AGA TCT TTA ATG GTT CAA GAT GAA TTC CGA C (SEQ ID NO: 29), the gene of Sp1814 was amplified from the genome of Sphingomonas sp. HXN-200. After double digestion by BspHI and BglII and ligation with pRSFduet, the resulting recombinant plasmid (pRSF-Sp1814) was successfully transformed into E. coli.

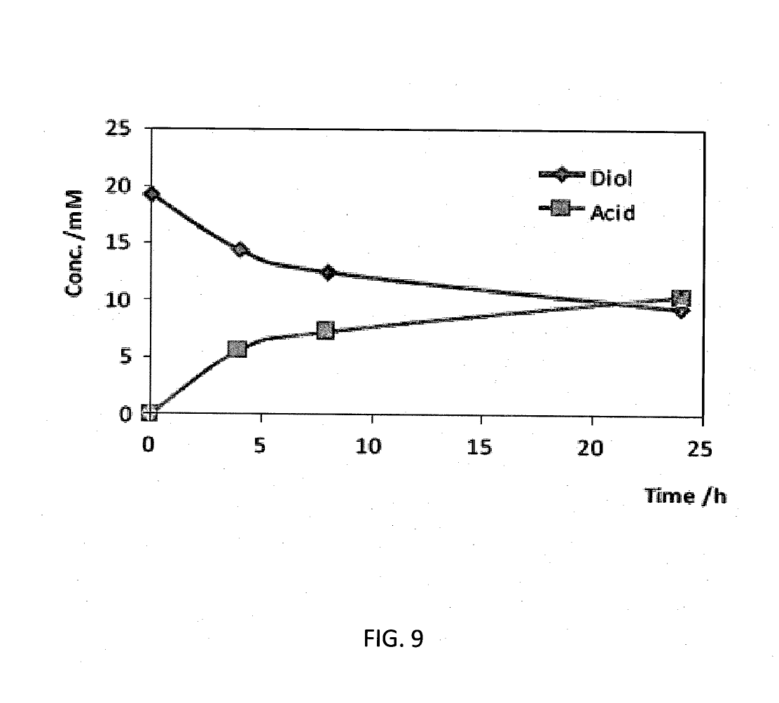

Two recombinant E. coli strains containing plasmid pRSF-Sp1814 and pET28a-alkH (Example 9) were grown in 1 mL LB medium containing 50 mg/L kanamycin and 50 mg/L and streptomycin 50 mg/L at 37° C. and then inoculated into 25 mL TB medium (50 mg/L kanamycin and 50 mg/L streptomycin). When OD₆₀₀ reached 0.6, 0.5 mM IPTG was added to induce the expression of protein. The cells continued to grow for 5 hours at 30° C. before they were harvested by centrifuge (5000 g, 5 mins). The cells were resuspended in 100 mM KPB buffer (pH=8.0) to 5 g cdw/L each and the substrate phenylethane-1,2-diol (20 mM) was loaded to start the reaction. The reaction was conducted at 30° C. and 300 rpm in a 100-mL flask for 24 hours. A 100 uL aqueous sample was taken during the reaction and analyzed by reverse phase HPLC (Agilent poroshell 120 SB-C18 column, acetonitrile:water:trifluoroacetic acid=30:70:0.1, flow rate 0.5 mL/min) to quantify the conversion of diol to acid. In 24 hours, about 10 mM mandelic acid was produced (FIG. 9). These results show that the Sp1814 alcohol dehydrogenase from Sphingomonas sp. HXN-200 is also useful for oxidation of diols to α-hydroxy carboxylic acids.

### Example 8

### Production of Mandelic Acid from Phenylethane-1,2-Diol Via Oxidation by Wild Type Acetic Acid Bacterium

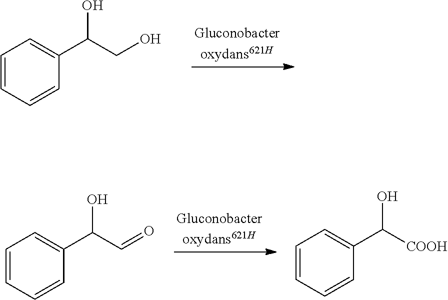 [SMILES: OCC(O)c1ccccc1 ; O=CC(O)c1ccccc1 ; O=C(O)C(O)c1ccccc1]

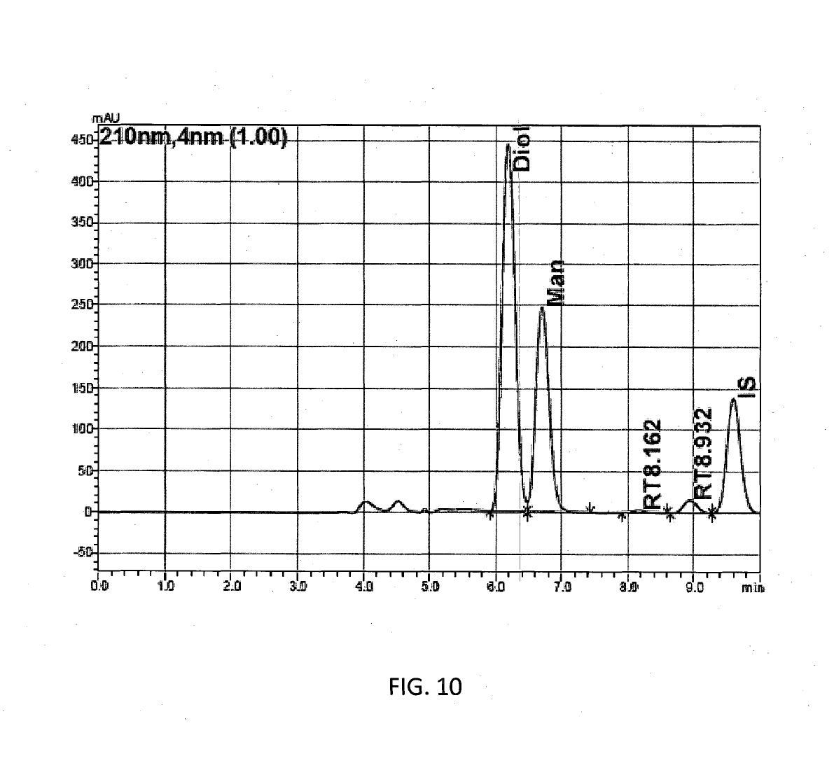

Acetic acid bacteria have a powerful enzyme system for oxidation of alcohols to acids with many potential industrial applications. We investigated whether acetic acid bacteria can convert diol to α-hydroxy carboxylic acids. Gluconobacter oxydans 621H was chosen as a model acetic acid bacterium because of commercial availability (from ATCC) of this strain and the well-known genetic background. The Gluconobacter oxydans 621H was inoculated in 50 mL glycerol medium at 30° C. to grow for 24 hours. Then, the cells were harvested by centrifuge (5000 g, 5 mins). The cells were resuspended in 100 mM KPB buffer (pH=8.0) to 5 g cdw/L each and the substrate phenylethane-1,2-diol (20 mM) was loaded to start the reaction. The reaction was conducted at 30° C. and 300 rpm in a 100-mL flask for 24 hours. A 100 uL aqueous sample was taken during the reaction and analyzed by reverse phase HPLC (Agilent poroshell 120 SB-C18 column, acetonitrile:water:trifluoroacetic acid=30:70:0.1, flow rate 0.5 mL/min) to quantify the conversion of diol to acid. In 6 hours, about 7 mM mandelic acid was produced (FIG. 10). These results show that wild type acetic acid bacteria (such as Gluconobacter oxydans 621H) are also useful biocatalysts for oxidation of diols to α-hydroxy carboxylic acids, which can be combined with recombinant cells to achieve the asymmetric one-pot transformation of alkenes to α-hydroxy carboxylic acids.

### Example 9

### Production of (S)-Mandelic Acid from Styrene Via Cascade Biocatalysis by Using E. coli Cells Co-Expressing SMO, SpEH and AlkJ

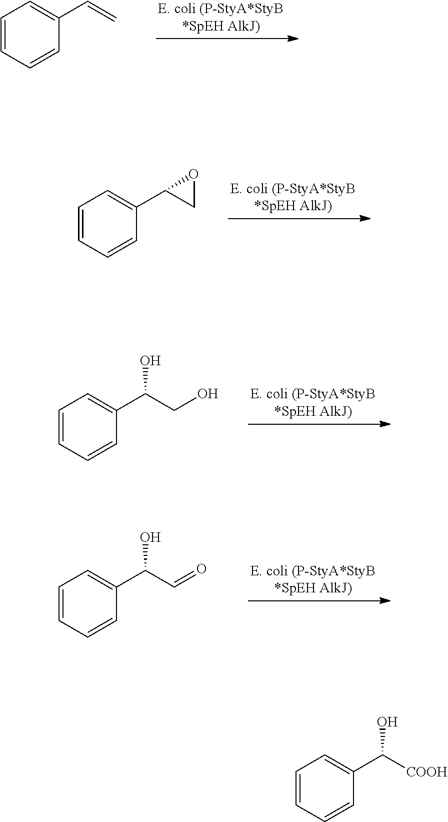

To produce α-hydroxy carboxylic acids, alcohol dehydrogenase was used to oxidize the diol intermediates. The alcohol dehydrogenase alkJ from the alkane degradation strain Pseudomonas putida was cloned from the OCT plasmid to E. coli pET28a plasmid (from Novagen) at the restriction site BamHI and SalI using primers CGC GGA TCC ATG TAC GAC TAT ATA ATC GTT GGT G (SEQ ID NO: 30) and CGC GTC GAC TTA CAT GCA GAC AGC TAT CAT GGC (SEQ ID NO: 31). The plasmid was successful transformed in to competent E. coli to produce the recombinant cells that successfully catalyzed (S)-phenylethane-1,2-diol to (S)-mandelic acid. In order to perform the multistep reactions inside one cell, these pET28a-alkJ and pCDF-StyAB*SpEH (which was subcloned from plasmid pRSF-StyAB*SpEH) were also transformed together into one E. coli cell (T7 expression strain from NEB and BL21DE3 strain from Novagen) to produce E. coli cells co-expressing SMO, SpEH and alkJ.

The recombinant E. coli strain containing the plasmid pCDF-StyAB*SpEH and the plasmid pET28a-AlkJ was grown in 1 mL LB medium containing 50 mg/L kanamycin and 50 mg/L and streptomycin 50 mg/L at 37° C. and then inoculated into 25 mL TB medium (50 mg/L kanamycin and 50 mg/L streptomycin). When OD₆₀₀ reached 0.6, 0.5 mM IPTG was added to induce the expression of protein. The cells continued to grow for 5 hours at 30° C. before they were harvested by centrifuge (5000 g, 5 mins). The cells were resuspended in 100 mM KPB buffer (pH=8.0) to 10 g cdw/L and mixed with hexadecane to form an aqueous:organic two-phase system (2 mL:2 mL). The styrene loading was 100 mM (10.4 g/L). The reaction was conducted at 30° C. and 300 rpm in a 100-mL flask for 8 hours, and a 100 uL aqueous sample was taken during the reaction and analyzed by reverse phase HPLC (Agilent poroshell 120 SB-C18 column, acetonitrile:water:trifluoroacetic acid=30:70:0.1, flow rate 0.5 mL/min) to quantify the production of diols and acids. At 8 hours, about 2 mM (0.3 g/L) (S)-mandelic acid were produced. These results show that the recombinant cells containing SMO, SpEH and AlkJ are capable catalysts for the cascade transformation of styrene to (S)-mandelic acid, and prove the feasibility of practice of our invented novel cascade biocatalysis route to enantiomerically pure α-hydroxy carboxylic acids from terminal alkenes.

### Example 10

### Production of (S)-Mandelic Acid from Styrene Via Cascade Biocatalysis Using E. coli Cells Co-Expressing SMO and SpEH, E. coli Cells Expressing AlkJ, and E. coli Cells Expressing AlkH

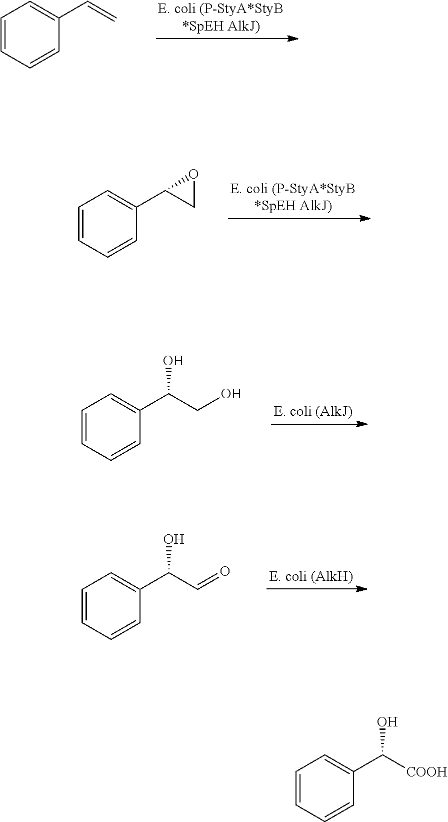

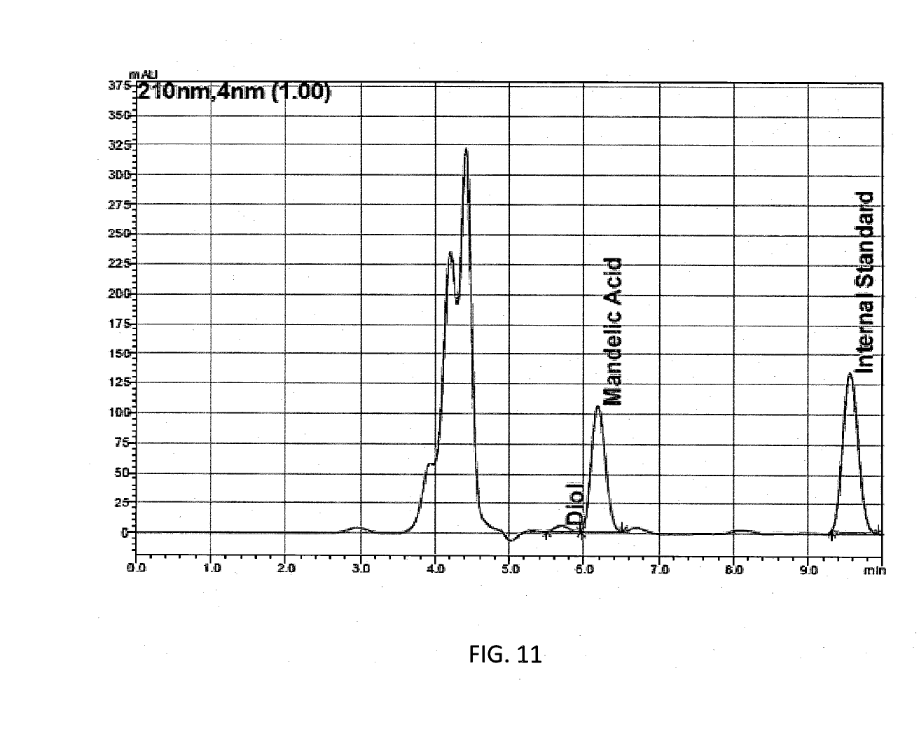

AlkH, an aldehyde dehydrogenase from alkane degradation strain Pseudomonas putida, was cloned from the OCT plasmid to E. coli pET28a plasmid (from Novagen) at the restriction site NdeI and XhoI using primers A TTC CAT ATG ACC ATA CCA ATT AGC CTA GCC A (SEQ ID NO: 32) and CCG CTC GAG TCA GCT CAA ATA CTT AAC TGT GAT AC (SEQ ID NO: 33). The recombinant E. coli cell containing this plasmid pET28a-alkH expressed the alkH well. Three recombinant E. coli strains, containing plasmid pRSFduet-StyAB*SpEH (Example 1), pET28a-alkJ (Example 9), and pET28a-alkH (in this Example), respectively, were grown separately in 1 mL LB medium containing 50 mg/L kanamycin at 37° C. and inoculated into 25 mL TB medium (50 mg/L kanamycin). When OD₆₀₀ reached 0.6, 0.5 mM IPTG was added to induce the expression of proteins. The cells continued to grow for another 5 hours at 30° C. before they were harvested by centrifuge (5000 g, 5 mins). The cells were resuspended in 5 mL 100 mM KPB buffer (pH=8.0) and OD₆₀₀ was measured. These cells were employed as multi-cell catalysts to transform styrene to (S)-mandelic acid in an aqueous system (2 mL 100 mM KPB buffer, pH=8.0). The cells were mixed with different loading: 2 g cdw/L for E. coli expressing SMO and SpEH, 5 g cdw/L for E. coli (alkJ), and 10 g cdw/L for E. coli (alkH) recombinant cells. The substrate (styrene) loading was 2 mM (prepared from 1M styrene stock solution in DMSO). The reaction was conducted at 30° C. and 300 rpm in a 100-mL flask for 24 hours, and a 100 uL aqueous sample was taken during the reaction and analyzed by reverse phase HPLC (Agilent poroshell 120 SB-C18 column, acetonitrile:water:trifluoroacetic acid=30:70:0.1, flow rate 0.4 mL/min) to quantify the production of diols and acids. In 5 hours, about 1.5 mM (S)-mandelic acid were produced (FIG. 11; yield 75%). These results show that the use of cells of multiple E. coli strains in one pot is an alternative way to carry out cascade transformation of styrene to synthesize (S)-mandelic acid. They confirm, once again, that our invented new cascade biocatalysis route to enantiomerically pure α-hydroxy carboxylic acids from terminal alkenes is feasible.

### Example 11

### Genetic Construction of Upstream Modules and Downstream Modules on Different Plasmids and Development of 12 Different E. coli Strains

The previous construction P-StyA*StyB*SpEH on pRSF (Example 1) was used as the template for genetic construction of upstream modules on the other three plasmids. The upstream module (StyAB*SpEH) was amplified using the primers A CTG TCA TGA AAA AGC GTATCG GTA TTG TTG G (SEQ ID NO: 17) and A TCG CTC GAG TCA AAG ATC CAT CTG TGC AAA GGC C (SEQ ID NO: 23) and then double digested by BspHI and XhoI. The vectors pACYC, pCDF, and pETduet (available from Novagen) were double digested by NcoI and XhoI and then ligated to the upstream module (StyAB*SpEH). The ligation DNA products were transformed into competent E. coli and selected on LB agar plates with appropriate antibiotics. The recombinant E. coli showed expression of SMO and SpEH on SDS-PAGE and activity towards styrenes. These results proved the construction of upstream module was successful.

To construct the downstream module (AlkJ*EcALDH), the gene of AlkJ was first amplified from pET28a-AlkJ (Example 9) using the primers A CTG GGA TCC GAT GTA CGA CTA TAT AAT CGT TGG TGC TG (SEQ ID NO: 34) and A CTG AGA TCT TTA CAT GCA GAC AGC TAT CAT GGC C (SEQ ID NO: 35) and then double digested by BamHI and BglII. The digested product was ligated to BamHI and BglII digested pRSF vector, transformed into competent E. coli, and selected on LB agar plate with kanamycin. The construction pRSF-AlkJ was then used as vector to insert the gene of EcALDH. The EcALDH gene was amplified by primers CG AGA TCT TAA GGA GAT ATA TAA TGA CAG AGC CGC ATG TAG CAG TAT TA (SEQ ID NO: 36) and A CTG CTC GAG TTA ATA CCG TAC ACA CAC CGA CTT AG (SEQ ID NO: 37) and then digested with BglII and XhoI. The EcALDH gene fragment was ligated to pRSF-AlkJ to give pRSF-AlkJ*EcALDH, which was the downstream module on pRSF plasmid. The downstream module was sub-cloned to three other plasmids, pACYC, pCDF, and pETduet, using the primers A CTG GGA TCC G AT GTA CGA CTA TAT AAT CGT TGG TGC TG (SEQ ID NO: 34) and A CTG CTC GAG TTA ATA CCG TAC ACA CAC CGA CTT AG (SEQ ID NO: 37), and the product was inserted on the BamHI/XhoI sites of the three plasmids. The recombinant E. coli showed expression of AlkJ and EcALDH on SDS-PAGE and activity towards phenyl ethane diol. These results proved the construction of downstream module was successful.

To develop the E. coli strains co-expressing four enzymes, the plasmids (pACYC, pCDF, pETduet, and pRSF) with upstream module and plasmids (pACYC, pCDF, pETduet, and pRSF) with downstream module were co transformed into competent E. coli cells. The E. coli cells were selected on LB agar plates with combination of appropriate antibiotics. The E. coli cells containing the two modules were grown in the media with two antibiotics. The 12 developed strains were E. coli (ACCD5), E. coli (ACET5), E. coli (ACRS5), E. coli (CDAC5), E. coli (CDET5), E. coli (CDRS5), E. coli (ETAC5), E. coli (ETCD5), E. coli (ETRS5), E. coli (RSAC5), E. coli (RSCD5), and E. coli (RSET5).

### Example 12

### Screening of 12 Different E. coli Strains for Efficient Oxidation of Styrene to S-Mandelic Acids

The recombinant E. coli strains containing both upstream module and downstream module were grown in 1 mL LB medium with combination of appropriate antibiotics at 37° C. and then inoculated into 15 mL M9 medium with 25 g/L glucose, 5 g/L yeast extract, and appropriate antibiotics. When OD₆₀₀ reached 0.6, 0.5 mM IPTG was added to induce the expression of protein. The cells continued to grow for 12 hours at 22° C. before they were harvested by centrifuge (5000 g, 10 mins) The cells were washed with 200 mM KP buffer (pH=8.0) and then resuspended to 10 g cdw/L. The resting cells were mixed with n-hexadecane to form an aqueous:organic two-phase system (2 mL:2 mL) containing 0.5% glucose. The styrene loading was 100 mM (10.4 g/L). The reaction was conducted at 30° C. and 300 rpm in a 100-mL flask for 20 hours, and a 100 uL aqueous sample was taken during the reaction and analyzed by reverse phase HPLC (Agilent poroshell 120 SB-C18 column, acetonitrile:water:trifluoroacetic acid=30:70:0.1, flow rate 0.5 mL/min) to quantify the production of diols and acids. At 20 hours, various concentrations of (S)-mandelic acid were produced, from 20 to 83 mM (FIG. 13). To further verify the results, three sets of independent experiments were performed. The results showed the necessity of optimizing the enzyme expression level in whole cell catalyst. The best strain, E. coli (ACRS5), efficiently produced 83±7 mM (S)-mandelic acid from 100 mM styrene in 20 hours. This not only proves the feasibility of our invented novel cascade biocatalysis route to enantiomerically pure α-hydroxy carboxylic acids from terminal alkenes, but also provides a good starting point to further optimize and improve the process to meet the industrial requirement.

### Example 13

### Optimization of the Cascade Oxidation of Styrene to S-Mandelic Acids by E. coli (ACRS5)

Several reaction parameters were explored to achieve the optimized system for the cascade biocatalysis. The substrate loading was increased from 100 to 120 and 150 mM. The cell density was varied from 10 to 20 g cdw/L. And the most important factor, glucose concentration, was investigated. The typical setup of the reaction system was similar to those in the Example 12: 200 mM KP buffer (pH=8.0):n-hexadecane (2 mL:2 mL) After intensive investigation, two key points were found: (1) increasing the cell loading will help the reaction, but too high density of cells will cost more than gains; (2) glucose is good for providing the NADH cofactor for the first step epoxidation by SMO, but it will also inhibit the oxidation of diol to aldehyde and acid. The optimal conditions were cell loading at 15 g cdw/L and glucose loading at 0.25% w/v. Under these optimal conditions, E. coli (ACRS5) produced 94±2 mM (about 14.3 g/L) (S)-mandelic acid from 120 mM styrene in 22 hours (FIG. 14). The ee of the (S)-mandelic acid was determined to be >98% by chiral HPLC. The intermediate diol was at the low level of 9 mM, and one byproduct, phenylethanol, was at the low concentration of 12 mM. These results show the applicable potential of the cascade biocatalysis; further investigation, such as using growing cells to perform the reaction in fermentor and in situ removal of the mandelic acid by ion exchange resin, will further improve the concentration and productivity.

### Example 14

### Cascade Oxidation of Substituted Styrene to Substituted S-Mandelic Acids by E. coli (ACRS5)

The recombinant E. coli (ACRS5) was grown in 1 mL LB medium with 50 mg/L kanamycin and 50 mg/L chloramphenicol at 37° C. and then inoculated into 25 mL M9 medium with 25 g/L glucose, 5 g/L yeast extract, and 50 mg/L kanamycin and 50 mg/L chloramphenicol. When OD₆₀₀ reached 0.6, 0.5 mM IPTG was added to induce the expression of protein. The cells continued to grow for 12 hours at 22° C. before they were harvested by centrifuge (5000 g, 10 mins). The cells were washed with 200 mM KP buffer (pH=8.0) and then resuspended to 10 g cdw/L. The resting cells were mixed with n-hexadecane to form an aqueous:organic two-phase system (2 mL:2 mL) containing 0.5% glucose. The substituted styrenes were added at 20 mM. The reaction was conducted at 30° C. and 300 rpm in a 100-mL flask for 12 hours, and a 100 uL aqueous sample was taken during the reaction and analyzed by reverse phase HPLC (Agilent poroshell 120 SB-C18 column, acetonitrile:water:trifluoroacetic acid=30:70:0.1, flow rate 0.5 mL/min) to quantify the production of diols and acids. The acid product was also extracted out by adding HCl and ethyl acetate. The ethyl acetate was removed and the residues were analyzed by chiral HPLC to determine the ee value. The results are summarized in Table 4.

TABLE 4
Conversion of styrene derivatives to substitued (S)-MA by E. coli (ACRS5)^{[a]}

| | Activity | Conversion | | Yield | ee (%) |
|---|---|---|---|---|---|
| Substrate | (U/g cdw)^{[b]} | (%)^{[c]} | Product | (%)^{[c]} | (configuration)^{[d]} |
| 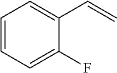 | 13 | 95 | 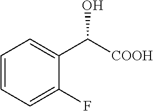 [SMILES: O=C(O)[C@@H](O)c1ccccc1F] | 92 | N.D |
| 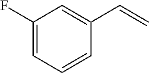 | 22 | 98 | 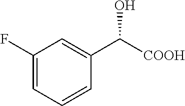 [SMILES: O=C(O)[C@@H](O)c1cccc(F)c1] | 95 | 98.4(S) |
| 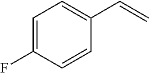 | 21 | 98 | 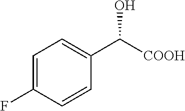 [SMILES: O=C(O)[C@@H](O)c1ccc(F)cc1] | 98 | N.D. |
| 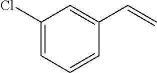 | 5 | 88 | 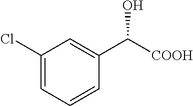 [SMILES: O=C(O)[C@@H](O)c1cccc(Cl)c1] | 83 | 96.6(S) |
| 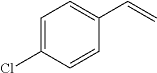 | 5 | 78 | 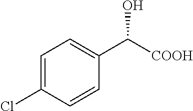 [SMILES: O=C(O)[C@@H](O)c1ccc(Cl)cc1] | 73 | N.D. |
| 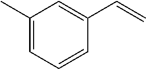 | 8 | 97 | 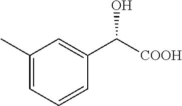 | 72 | 98.4(S) |

^{[a]}The reaction was performed in a two-phase system consisting of KPB buffer (200 mM, pH 8.0, containing 0.5% glucose and 10 g cdw/L cells) and n-hexadecane (1:1) with 20 mM substrate for 12 hours.
^{[b]}Activity was determined at initial 60 min.
^{[c]}Conversion and yield were determined by HPLC analysis.
^{[d]}ee value was determined by chiral HPLC analysis.

In general, the reactions were quite efficient because of the high conversions (78-98%) of the six substituted styrenes and the high yield (72%-98%) of the final product substituted (S)-mandelic acids. Furthermore, the accumulation of diol intermediates or by-products was either insignificant (<10%) or not observed (<1%). The enantiomeric excess of three of the substituted (S)-mandelic acids was determined to be 96.6-98.4%. This proves the cascade biocatalytic process is highly enantioselective with relatively broad substrate scope.

### Example 15

### Production of (1R, 2R) Aryl Cyclic Diols from Olefins Via Cascade Biocatalysis Using E. coli Cells Expressing SMO and SpEH or StEH

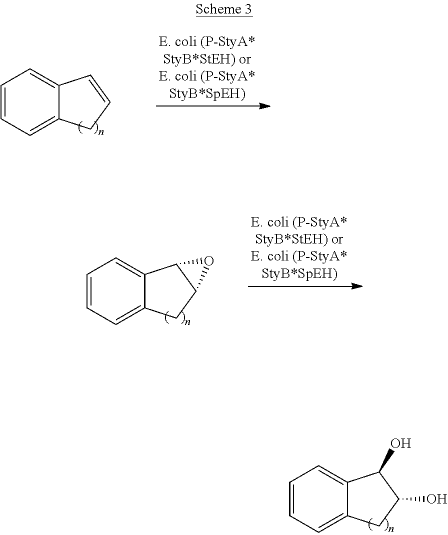

To research the potential for production of aryl cyclic diols from olefins, we then tested the E. coli (P-StyA*StyB*StEH) and E. coli (P-StyA*StyB*SpEH) for dihydroxylation of indene and 1,2-dihydronaphthalene. The E. coli (P-StyA*StyB*StEH) and E. coli (P-StyA*StyB*SpEH) were grown in 1 mL LB medium containing 50 mg/L kanamycin at 37° C. and then 2% inoculated into 25 mL M9-Glu-Y medium with 50 mg/L kanamycin. When OD₆₀₀ reached 0.6, 0.5 mM IPTG was added to induce the expression of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL:2 mL) for biotransformation of 20 mM indene and 1,2-dihydronaphthalene.

TABLE 5
Conversion of cyclic olefins to cyclic diols by E. coli (P-StyA*StyB*StEH) and E. coli
(P-StyA*StyB*SpEH)^{[a]}

| | | Activity | Conversion | | Yield | ee | de |
|---|---|---|---|---|---|---|---|
| Catalyst | Substrate | (U/g cdw)^{[b]} | (%)^{[c]} | Product | (%)^{[c]} | (%) | (%)^{[d]} |
| E. coli (P-StyA* StyB*SpEH) | 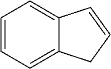 | 28 | 97 | 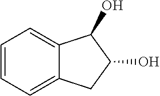 [SMILES: O[C@@H]1Cc2ccccc2[C@H]1O] | 80 | 98.0 | 98.8 |
| E. coli (P-StyA* StyB*StEH) | 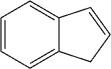 | 20 | 98 | 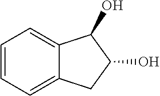 [SMILES: O[C@@H]1Cc2ccccc2[C@H]1O] | 71 | 96.1 | 98.1 |
| E. coli (P-StyA* StyB*SpEH) | 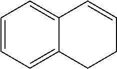 | 4 | 75 | 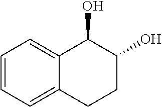 [SMILES: O[C@@H]1CCc2ccccc2[C@H]1O] | 73 | 96.8 | >99 |
| E. coli (P-StyA* StyB*StEH) | 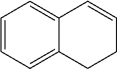 | 4 | 69 | 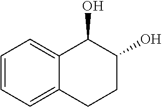 [SMILES: O[C@@H]1CCc2ccccc2[C@H]1O] | 67 | 99.6 | >99 |

^{[a]}The reaction was performed in a two-phase system consisting of KPB buffer (200 mM, pH 8.0, containing 0.5% glucose and 10 g cdw/L cells) and n-hexadecane (1:1) with 20 mM substrate for 8 hours.
^{[b]}Activity was determined at initial 60 min.
^{[c]}Conversion and yield were determined by HPLC analysis.
^{[d]}ee and de value was determined by chiral HPLC analysis.

The reaction was conducted at 30° C. and 300 rpm in a 100-mL flask for 8 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. The ee of the product diols was determined by chiral HPLC. As can be seen in Table 5, two (1R, 2R)-diols can be produced in high ee (>96% ee) and very high de (>98% ee) with good yields (>67%) by E. coli (P-StyA*StyB*SpEH) or E. coli (P-StyA*StyB*StEH) cells. The recombinant biocatalysts E. coli (P-StyA*StyB*StEH) and E. coli (P-StyA*StyB*SpEH) were proven to accept cyclic styrene analogues and give (1R, 2R)-cyclic diols as valuable products.

### Example 16

### Production of Four Enantiomers of 1-Phenyl-1,2-Propanediol from β-Methyl Styrenes Via Cascade Biocatalysis Using E. coli Cells Expressing SMO and SpEH or StEH

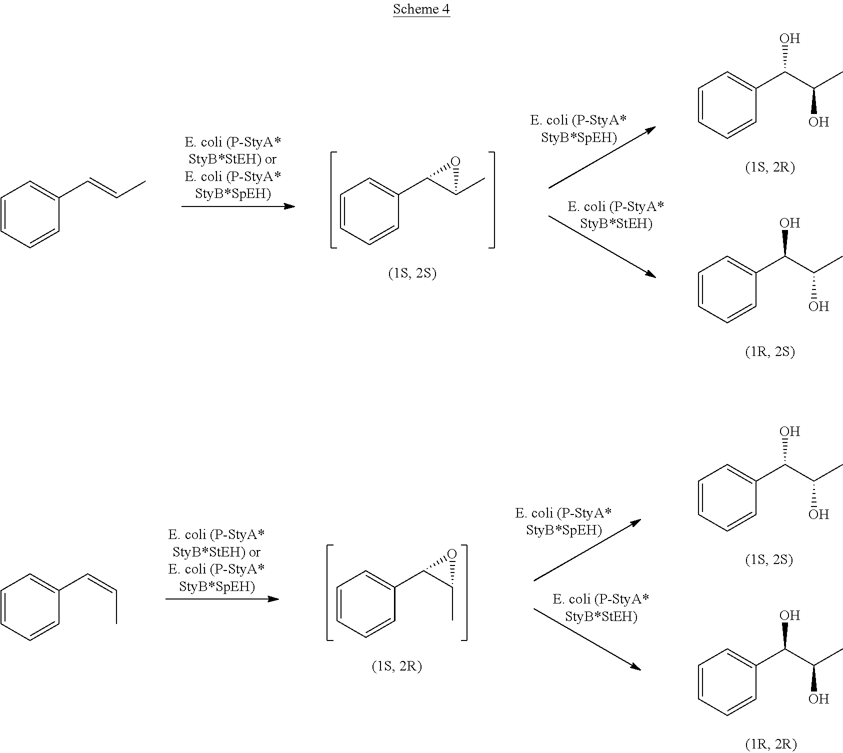

To research the potential for production of nonterminal aryl diols from olefins, we then tested the E. coli (P-StyA*StyB*StEH) and E. coli (P-StyA*StyB*SpEH) for dihydroxylation of two different forms of β-methyl styrenes. The E. coli (P-StyA*StyB*StEH) and E. coli (P-StyA*StyB*SpEH) were grown in 1 mL LB medium containing 50 mg/L kanamycin at 37° C. and then 2% inoculated into 25 mL M9-Glu-Y medium with 50 mg/L kanamycin. When OD₆₀₀ reached 0.6, 0.5 mM IPTG was added to induce the expression of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL:2 mL) for biotransformation of 20 mM β-methyl styrenes.

TABLE 6
Conversion of β-methyl styrenes to 1-phenyl-1,2-propanediol by E. coli (P-StyA*StyB*StEH) and E. coli
(P-StyA*StyB*SpEH)^{[a]}

| | | Activity | Conversion | | Yield | ee | de |
|---|---|---|---|---|---|---|---|
| Catalyst | Substrate | (U/g cdw)^{[b]} | (%)^{[c]} | Product | (%)^{[c]} | (%) | (%)^{[d]} |
| E. coli (P-StyA* StyB*SpEH) | 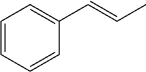 | 23 | 80 | 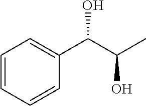 | 22 | 94 | 91.8 |
| E. coli (P-StyA* StyB*StEH) | 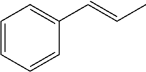 | 15 | 99 | 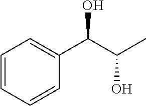 | 96 | >98 | 98.0 |
| E. coli (P-StyA* StyB*SpEH) | 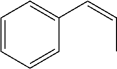 | 19 | 94 | 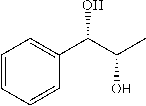 | 75 | 85.6 | >99 |
| E. coli (P-StyA* StyB*StEH) | 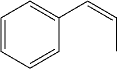 | 20 | 97 | 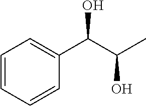 | 89 | 98.8 | >99 |

^{[a]}The reaction was performed in a two-phase system consisting of KPB buffer (200 mM, pH 8.0, containing 0.5% glucose and 10 g cdw/L cells) and n-hexadecane (1:1) with 20 mM substrate for 8 hours.
^{[b]}Activity was determined at initial 60 min.
^{[c]}Conversion and yield were determined by HPLC analysis.
^{[d]}ee and de value was determined by chiral HPLC analysis.

The reaction was conducted at 30° C. and 300 rpm in a 100-mL flask for 8 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. The ee of the product diols was determined by chiral HPLC. As can be seen in Table 6, four enantiomers of 1-phenyl-1,2-propanediol, including (1S, 2R), (1R, 2S), (1S, 2S), and (1R, 2R), can be produced from two different β-methyl styrenes by E. coli (P-StyA*StyB*SpEH) and E. coli (P-StyA*StyB*StEH) cells. The recombinant biocatalysts E. coli (P-StyA*StyB*SpEH) and E. coli (P-StyA*StyB*StEH) are stereo-complementary whole cell catalysts for trans-dihydroxylation of nonterminal styrene analogues.

### Example 17

### Production of Other Aryl Vicinal Diols from Aryl Olefins Via Cascade Biocatalysis Using E. coli Cells Expressing SMO and SpEH or StEH

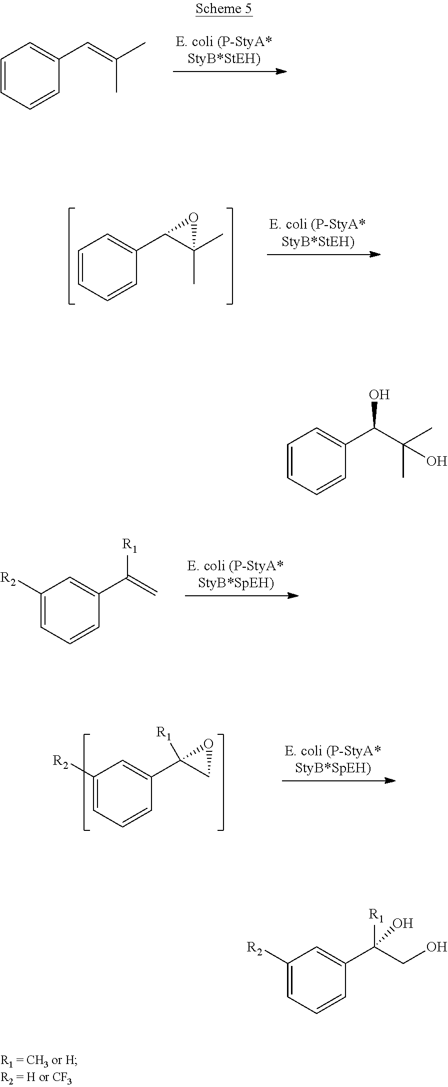

To research the potential for production of other aryl vicinal diols from olefins, we then tested the E. coli (P-StyA*StyB*StEH) and E. coli (P-StyA*StyB*SpEH) for dihydroxylation of 2-methyl-1-phenyl-1-propene and α-methylstyrene. The E. coli (P-StyA*StyB*StEH) and E. coli (P-StyA*StyB*SpEH) were grown in 1 mL LB medium containing 50 mg/L kanamycin at 37° C. and then 2% inoculated into 25 mL M9-Glu-Y medium with 50 mg/L kanamycin. When OD₆₀₀ reached 0.6, 0.5 mM IPTG was added to induce the expression of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used in a buffer:hexadecane two-phase system (2 mL:2 mL) for biotransformation of 20 mM 2-methyl-1-phenyl-1-propene and α-methylstyrene.

TABLE 7
Conversion of 2-methyl-1-phenyl-1-propene and α-methylstyrene by E. coli (P-StyA*StyB*StEH)
and E. coli (P-StyA*StyB*SpEH)^{[a]}

| | | Activity | Conversion | | Yield | ee |
|---|---|---|---|---|---|---|
| Catalyst | Substrate | (U/g cdw)^{[b]} | (%)^{[c]} | Product | (%)^{[c]} | (%)^{[d]} |
| E. coli (P-StyA* StyB*SpEH) | 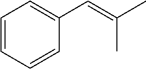 | 16 | 76 | 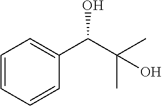 | 11 | 3.4 |
| E. coli (P-StyA* StyB*StEH) | 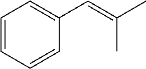 | 17 | 80 | 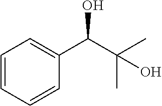 | 83 | 98.2 |
| E. coli (P-StyA* StyB*SpEH) | 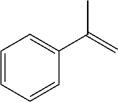 | 8 | 62 | 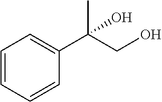 | 56 | 94.5 |
| E. coli (P-StyA* StyB*StEH) | 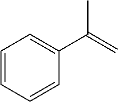 | 11 | 68 | 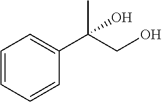 | 24 | 46.8 |

^{[a]}The reaction was performed in a two-phase system consisting of KPB buffer (200 mM, pH 8.0, containing 0.5% glucose and 10 g cdw/L cells) and n-hexadecane (1:1) with 20 mM substrate for 8 hours.
^{[b]}Activity was determined at initial 60 min.
^{[c]}Conversion and yield were determined by HPLC analysis.
^{[d]}ee and de value was determined by chiral HPLC analysis.

The reaction was conducted at 30° C. and 300 rpm in a 100-mL flask for 8 hours. A 100 uL aqueous sample was taken during the reaction and analyzed by reverse phase HPLC (Agilent poroshell 120 EC-C18 column, acetonitrile:water=60:40, flow rate 0.5 mL/min) to quantify the production of diols. The ee of the product diols was determined by chiral HPLC. As can be seen in Table 7, (R)-1-phenyl-2-methyl-1,2-propanediol was produced in high ee from 2-methyl-1-phenyl-1-propene by E. coli (P-StyA*StyB*StEH), and (S)-2-phenyl-1,2-propanediol was produced in high ee from α-methylstyrene by E. coli (P-StyA*StyB*SpEH) cells.

### Example 18

### 300 mg Scale Preparation of Aryl Vicinal Diols in High ee Via Cascade Biocatalysis Using E. coli Cells Expressing SMO and SpEH or StEH

To further demonstrate the synthetic potential of trans-dihydroxylation via cascade biocatalysis, we carried out the preparation of 10 valuable vicinal diols from 7 aryl olefins using E. coli (P-StyA*StyB*StEH) and E. coli (P-StyA*StyB*SpEH). The E. coli (P-StyA*StyB*StEH) and E. coli (P-StyA*StyB*SpEH) were grown in 2 mL LB medium containing 50 mg/L kanamycin at 37° C. and then 2% inoculated into 200 mL M9-Glu-Y medium with 50 mg/L kanamycin. When OD₆₀₀ reached 0.6, 0.5 mM IPTG was added to induce the expression of enzymes. The cells continued to grow and expressed protein for 12 hours at 22° C. before they were harvested by centrifuge (5000 g, 5 mins) The cells were resuspended in 100 mM KPB buffer (pH=8.0) to 20 g cdw/L and used in a buffer:hexadecane two-phase system (45 mL:5 mL) for biotransformation of 50 mM substrates.

TABLE 8
Preparation of (R)- or (S)- vicinal diols in high ee by enantioselective dihydroxylation of aryl alkenes
with resting cells of E. coli (P-StyA*StyB*StEH) and E. coli (P-StyA*StyB*SpEH)^{[a]}

| | | Time | | Isolated | Yield | ee | de |
|---|---|---|---|---|---|---|---|
| Substrate | Catalyst | (h) | Product | (g) | (%) | (%)^{b} | (%)^{c} |
| 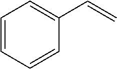 | E. coli (P-StyA*StyB* SpEH) | 5 | 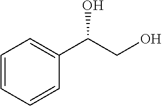 | 0.295 | 85.5 | 96.3 | n.a.^{d} |
| 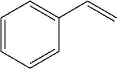 | E. coli (P-StyA*StyB* StEH) | 5 | 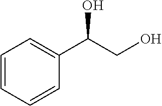 | 0.289 | 83.8 | 95.8 | n.a. |
| 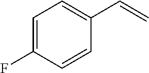 | E. coli (P-StyA*StyB* SpEH) | 6 | 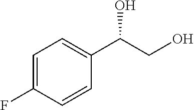 [SMILES: OC[C@@H](O)c1ccc(F)cc1] | 0.299 | 76.7 | 96.7 | n.a. |
| 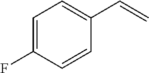 | E. coli (P-StyA*StyB* StEH) | 5 | 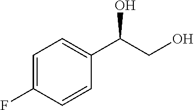 [SMILES: OC[C@H](O)c1ccc(F)cc1] | 0.325 | 80.7 | 96.7 | n.a. |
| 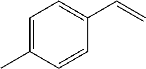 | E. coli (P-StyA*StyB* SpEH) | 8 | 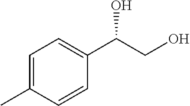 | 0.279 | 73.4 | 92.4 | n.a. |
| 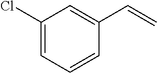 | E. coli (P-StyA*StyB* SpEH) | 8 | 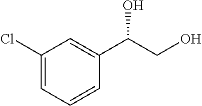 [SMILES: OC[C@@H](O)c1cccc(Cl)c1] | 0.326 | 75.6 | 96.5 | n.a. |
| 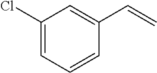 | E. coli (P-StyA*StyB* StEH) | 8 | 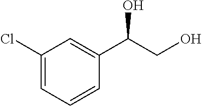 [SMILES: OC[C@H](O)c1cccc(Cl)c1] | 0.304 | 70.6 | 96.3 | n.a. |
| 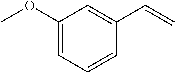 | E. coli (P-StyA*StyB* SpEH) | 6 | 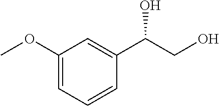 | 0.358 | 85.3 | 96.8 | n.a. |
| 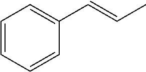 | E. coli (P-StyA*StyB* StEH) | 7 | 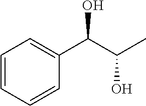 | 0.313 | 82.3 | >98 | 98.2 |
| 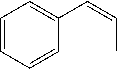 | E. coli (P-StyA*StyB* StEH) | 8 | 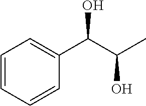 | 0.300 | 78.8 | 98.6 | >99 |

^{[a]}The reactions were performed with substrates (50 mM based on total volume) and resting cells (20 g
cdw/L) in a two-liquid phase system (50 mL) consisting of KP buffer (200 mM, pH 8.0, 2% glucose) and
n-hexadecane (9:1) at 30° C.
^{b}ee value was determined by chiral HPLC analysis.
^{c}de value was determined by chiral HPLC analysis.
^{d}n.a.: not applicable.

The reaction was conducted at 30° C. and 300 rpm in a 100-mL flask for 5-8 hours. The reaction was monitored by TLC. Once the substrate disappeared totally, the reaction mixture was then saturated with NaCl. After centrifugation, the aqueous phase was collected and washed with 10 mL n-hexane. The aqueous phase was then extracted with ethyl acetate three times (3×50 mL), and all the organic phases were combined. After drying over Na₂SO₄, the solvents were removed by evaporation. The crude diol products were purified by flash chromatography on a silica gel column with n-hexane:ethyl acetate (2-1:1) as eluent (R_{f}≈0.3 for all diol products). As can be seen in Table 8, all 10 useful and valuable vicinal diols were obtained in high ee (92.4-98.6%) and de (de≧98%, if applicable) with good isolated yield (70.6-85.5%). The final diol product was further verified by performing H-NMR and chiral HPLC analysis.

### Example 19

### Scaling Up the Cascade Biocatalysis for Production of (R)-Phenylethane-1,2-Diol in Bioreactor

The E. coli (P-StyA*StyB*StEH) was cultured in LB medium (2 mL) containing kanamycin (50 mg/L) at 37° C. for 7-10 hrs and then inoculated into 100 mL M9 medium containing glucose (30 g/L), yeast extract (5 g/L), and kanamycin (50 mg/L). The cells were grown at 30° C. for 12 hrs to reach an OD₆₀₀ of 15. All culture was transferred into 900 mL sterilized modified Riesenberg medium (containing: 13.3 g KH₂PO₄, 4.0 g (NH₄)₂HPO₄, 1.7 g Citric acid, 1.2 g MgSO₄.7H₂O, 4.5 mg Thiamin HCl, 15 g Glucose, 10 mL trace metal solution (6 g/L Fe(III) citrate, 1.5 g/L MnCl₂.4H₂O, 0.8 g/L Zn(CH₃COO)₂.2H₂O, 0.3 g/L H₃BO₃, 0.25 g/L Na₂MoO₄.2H₂O, 0.25 g/L CoCl₂.6H₂O, 0.15 g/L CuCl₂.2H₂O, 0.84 g/L EDTA, 0.1 M HCl)) with 15 g/L glucose as carbon source in a 3 L fermentor (Sartorius). The cells were grown in the fermentor at 30° C. for 12 hrs to reach an OD₆₀₀ of 15-18. During the batch growth, the pH value was maintained at 7.0 by adding 30% phosphoric acid or 25% ammonia solution based on pH sensing, the stirring rate was kept constant at 1000 rpm, and aeration rate was kept constant at 1 L/min. At the end of batch growth (12 hrs), PO₂ started to increase, indicating glucose depletion. Fed-batch growth was started by feeding a solution containing 730 g/L glucose and 19.6 g/L MgSO₄.7H₂O. The feeding rate was increased stepwise: 6.5 mL/hr for 1 hr, 8 mL/hr for 1 hr, 10 mL/hr for 1 hr, 13 mL/hr for 1 hr, then kept at 16 mL/hr until the end of reaction. Stirring rate was increased stepwise: 1200 rpm for 2 hrs, 1500 rpm for 2 hrs, then kept at 2000 rpm until the end of reaction. Aeration rate was increased stepwise: 1.2 L/min for 2 hrs, 1.5 L/min for 2 hrs, then kept at 2.0 L/min until the end of reaction. Antifoam PEG2000 (Fluka) was added when necessary. After fed-batch growth for 2 hrs, IPTG (0.5 mM) was added to induce the expression of protein. After fed-batch growth for 5 hrs, the cell density reached 20 g cdw/L, and the biotransformation started by adding styrene dropwise at the rate of 6 mL/hr for 4 hrs, and then 3 mL/hr for an additional 1 hr. The reaction was monitored by taking a sample every hour for analyzing the formation of (R)-phenylethane-1,2-diol by reverse phase HPLC. After 5 hrs of reaction, 120 mM (16.6 g/L) (R)-1-phenyl-1,2-ethanediol was produced in 96.2% ee with an average volumetric productivity of 3.3 g/L/hr for the reaction period.

### Example 20

### Production of 1-Hexene Oxide from 1-Hexene Using E. coli Cells Expressing P450pyrTM System

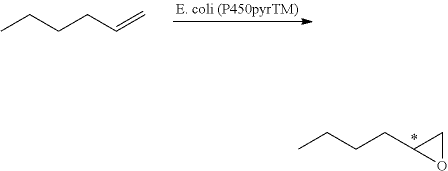

The genetic engineering of a recombinant E. coli strain expressing P450pyrTM system was done as described in Pham, S. Q. et al. Biotechnol. Bioeng. 110, 363-373 (2013). The resulting E. coli (P450pyrTM) was grown in 1 mL LB medium containing 50 mg/L kanamycin and 100 mg/L ampicillin at 37° C. and then 2% inoculated into 50 mL TB medium with 50 mg/L kanamycin and 100 mg/L ampicillin. When OD₆₀₀ reached 0.6, 0.5 mM IPTG and 0.5 mM ALA (S-Aminolevulinic acid hydrochloride) were added to induce the expression of enzymes. The cells continued to grow and expressed protein for 12 hours at 22° C. before they were harvested by centrifuge (5000 g, 5 mins). The cells were resuspended in 100 mM KPB buffer (pH=8.0) to 10 g cdw/L and used (4 mL) for biotransformation of 5 mM 1-hexene (1% ethanol as co-solvent and 1% glucose for cofactor regeneration). The reaction was conducted at 30° C. and 300 rpm in a 100-mL flask for 5 hours. After the reaction, the product was extracted by adding an equal amount of EtOAc containing 2 mM dodecane as the 2 mM docecane internal standard, the mixture was centrifuged at 1,5000 rpm for 10 mins, and the organic phase was dried over Na₂SO₄ and then subjected to chiral GC analysis for determination of product ee and conversion (Agilent 7890A gas chromatograph system with Macherey-Nagel FS-HYDRODEX β-TBDAc column 25 m×0.25 mm). The GC results showed that 1-hexene oxide was produced in 62% ee using E. coli (P450pyrTM). This demonstrates that the cascade biocatalysis route has potential for preparation of a broad scope of α-hydroxy carboxylic acids.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Also incorporated by reference in its entirety is Wu et al., ACS Catal. 4:409-20 (2014). Where any conflict exists between a document incorporated by reference and the present application, this application will control. All publically information associated with reference gene sequences disclosed in this application (such as SEQ ID NOs: 1-16), including, for example, genomic loci, genomic sequences, functional annotations, allelic variants, and reference mRNA (including, e.g., exon boundaries or response elements) and protein sequences (such as conserved domain structures, e.g., as identifiable by ENTREZ conserved domain searches or by multiple sequence alignments of homologous sequences), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

While this invention has been particularly shown and described with references to example embodiments thereof, it will be understood by those skilled in the art that various changes in form and details may be made therein without departing from the scope of the invention encompassed by the appended claims.

1. A composition comprising an alkene epoxidase and a selective epoxide hydrolase, wherein the composition is in the form of:
a) a recombinant microorganism expressing the alkene epoxidase and selective epoxide hydrolase;
b) a protein extract of the microorganism of a);
c) purified alkene epoxidase and purified selective epoxide hydrolase;
d) purified alkene epoxidase and purified selective epoxide hydrolase, wherein the purified enzymes are attached to solid supports;
e) a composition of any one of a)-d), further comprising a diol oxidation system; or
f) any combination of the foregoing.

2. A recombinant microorganism, comprising a first heterologous nucleic acid encoding an alkene epoxidase and a second heterologous nucleic acid encoding a selective epoxide hydrolase.

3. The recombinant microorganism of claim 2, wherein the alkene epoxidase is selected from a monooxygenase (such as styrene monooxygenase (such as StyAB), P450 monooxygenase, or alkene monooxygenase), lipase, or peroxidase.

4. The recombinant microorganism of claim 2, wherein the selective epoxide hydrolase is selected from an epoxide hydrolase from Sphingomonas, Solanum tuberosum, or Aspergillus, or a variant thereof that is at least 60% identical at the amino acid level to the epoxide hydrolase from Sphingomonas, Solanum tuberosum, or Aspergillus.

5. The recombinant microorganism of claim 2, further comprising a nucleic acid encoding a diol oxidation system.

6. The recombinant microorganism of claim 5, wherein the nucleic acid encoding a diol oxidation system is a heterologous nucleic acid.

7. The recombinant microorganism of claim 2, wherein the microorganism is a bacterium.

8. The recombinant microorganism of claim 7, wherein the bacterium is E. coli.

9. A composition comprising the recombinant microorganism of claim 2.

10. The composition of claim 1, further comprising a second recombinant microorganism comprising a nucleic acid encoding a diol oxidation system.

11. The composition of claim 10, wherein the numerical ratio of the first recombinant microorganism and second recombinant microorganism produces a relative maximum of yield of enantiomerically pure alpha-hydroxy carboxylic acid from an alkene.

12. The composition of claim 1, which is a liquid, preferably wherein the liquid is a two phase liquid comprising an aqueous phase and a second phase with improved solubility for an alkene relative to the aqueous phase.

13. The composition of claim 1, further comprising an alkene suitable for conversion to a diol or alpha carboxylic acid by the composition.

14. A method of non-toxic production of an enantiomerically pure vicinal diol, comprising contacting the composition of claim 1 with an alkene in a solution under conditions where the recombinant microorganism expresses the alkene epoxidase and selective epoxide hydrolase, thereby producing the enantiomerically pure vicinal diol, wherein the vicinal diol is produced from the alkene without intervening purification steps.

15. The method of claim 14, wherein the alkene is a terminal alkene, an aryl alkene, or an aryl terminal alkene.

16. The method of claim 15, wherein the alkene is any one of the substrates shown in any one of Tables 2-8 and Schemes 1-5, or a salt or ester thereof.

17. A method of non-toxic production of an enantiomerically pure alpha-hydroxy carboxylic acid, comprising contacting a terminal alkene in a solution with the composition of claim 1, under conditions where the recombinant microorganism expresses the alkene epoxidase and selective epoxide hydrolase and the diol oxidation system is expressed, thereby producing the enantiomerically pure alpha-hydroxy carboxylic acid, wherein the alpha-hydroxy carboxylic acid is produced from the terminal alkene without intervening purification steps.

18. The method of claim 17, wherein the terminal alkene is any one of the substrates shown in any one of Tables 2 and 3 and Schemes 1 and 2, or a salt or ester thereof.

19. The method of claim 14, wherein:
a) the yield is at least about: 30, 40, 50, 60, 65, 70, 75, 80, 85, 90, 91, 92, 93, 94, 95, 96, 97, 98, 99%, or more;
b) the enantiomeric excess (ee) is at least about: 30, 40, 50, 60, 65, 70, 75, 80, 85, 90, 91, 92, 93, 94, 95, 96, 97, 98, 99%, or more; or
c) both a) and b).

20. The method of claim 14, wherein the liquid solution is a two phase liquid comprising an aqueous phase and a second phase with improved solubility for an alkene relative to the aqueous phase.


SEQUENCE LISTING (37 sequences)

SEQ ID NO: 1 - protein, 415 aa, Pseudomonas sp. VLB120
  StyA
MKKRIGIVGAGTAGLHLGLFLRQHDVDVTVYTDRKPDEYSGLRLLNTVAHNAVTVQREVALDVNEWPSEEFGYFGHYYYVGGPQPMRFYGDLKAPSRAVDYRLYQPMLMRALEARGGKFCYDAVSAEDLEGLSEQYDLLVVCTGKYALGKVFEKQSENSPFEKPQRALCVGLFKGIKEAPIRAVTMSFSPGHGELIEIPTLSFNGMSTALVLENHIGSDLEVLAHTKYDDDPRAFLDLMLEKLGKHHPSVAERIDPAEFDLANSSLDILQGGVVPAFRDGHATLNNGKTIIGLGDIQATVDPVLGQGANMASYAAWILGEEILAHSVYDLRFSEHLERRRQDRVLCATRWTNFTLSALSALPPEFLAFLQILSQSREMADEFTDNFNYPERQWDRFSSPERIGQWCSQFAPTIAA

SEQ ID NO: 2 - protein, 170 aa, Pseudomonas sp. VLB120
  StyB
MTLKKDMAVDIDSTNFRQAVALFATGIAVLSAETEEGDVHGMTVNSFTSISLDPPTVMVSLKSGRMHELLTQGGRFGVSLLGESQKVFSAFFSKRAMDDTPPPAFTIQAGLPTLQGAMAWFECEVESTVQVHDHTLFIARVSACGTPEANTPQPLLFFASRYHGNPLPLN

SEQ ID NO: 3 - protein, 416 aa, Sphingomonas sp. HXN-200
  P450pyr
MEHTGQSAAATMPLDSIDVSIPELFYNDSVGEYFKRLRKDDPVHYCADSAFGPYWSITKYNDIMHVDTNHDIFSSDAGYGGIIIDDGIQKGGDGGLDLPNFIAMDRPRHDEQRKAVSPIVAPANLAALEGTIRERVSKTLDGLPVGEEFDWVDRVSIEITTQMLATLFDFPFEERRKLTRWSDVTTAAPGGGVVESWDQRKTELLECAAYFQVLWNERVNKDPGNDLISMLAHSPATRNMTPEEYLGNVLLLIVGGNDTTRNSMTGGVLALHKNPDQFAKLKANPALVETMVPEIIRWQTPLAHMRRTAIADSELGGKTIRKGDKVVMWYYSGNRDDEVIDRPEEFIIDRPRPRQHLSFGFGIHRCVGNRLAEMQLRILWEEILTRFSRIEVMAEPERVRSNFVRGYAKMMVRVHA

SEQ ID NO: 4 - protein, 440 aa, Rhodococcus coprophilus TC-2
  P450tol
MTTVESNTTAAIPDEIARQIVLPEGHKDNVPLFEAYRWLRENQPLGQARVEGYDPLWLITKYADLMEVERQPQIFAAGGGEDKGSNNPILANQAGDEFTRQLLGGNLRILDALPYLDQPEHSVVKDVAFDWFRPANLKKWEDRIRETARASIDRLLAGGPDLDAVQEFAVFFPLRVIMSLFGVPEEDEPRMMALTQDFFGVADPDAQRDDIEALSPDAAAQQWAATIADFYAYFDVLVESRRAEPRDDLATLIAVAKDENGEYFPKTFAYGWFVAIATAGHDTTASTLAGCLQSLAAHPEVLDRVKGDPDLIPDLVNESLRIVSPVKHFTRVALQDYEMRGQKIKAGDRLMLLFQSGNRDAEVFDRPDDFDIDRRPNKHIAFGYGPHMCIGQHLAKLELKVMLQELLPHLERVEVSGEPKLIQTNFVGGLRKLPVHLTFS

SEQ ID NO: 5 - protein, 381 aa, Sphingomonas sp. HXN-200
  SpEH
MMNVEHIRPFRVEVPQDALDDLRDRLARTRWPEKETVDDWDQGIPLAYARELAIYWRDEYDWRRIEARLNTWPNFLATVDGLDIHFLHIRSDNPAARPLVLTHGWPGSVLEFLDVIEPLSADYHLVIPSLPGFGFSGKPTRPGWDVEHIAAAWDALMRALGYDRYFAQGGDWGSAVTSAIGMHHAGHCAGIHVNMVVGAPPPELMNDLTDEEKLYLARFGWYQAKDNGYSTQQATRPQTIGYALTDSPAGQMAWIAEKFHGWTDCGHQPGGQSVGGHPEQAVSKDAMLDTISLYWLTASAASSARLYWHSFRQFAAGEIDVPTGCSLFPNEIMRLSRRWAERRYRNIVYWSEAARGGHFAAWEQPELFAAEVRAAFAQMDL

SEQ ID NO: 6 - protein, 321 aa, Solanum tuberosum
  StEH
MEKIEHKMVAVNGLNMHLAELGEGPTILFIHGFPELWYSWRHQMVYLAERGYRAVAPDLRGYGDTTGAPLNDPSKFSILHLVGDVVALLEAIAPNEEKVFVVAHDWGALIAWHLCLFRPDKVKALVNLSVHFSKRNPKMNVVEGLKAIYGEDHYISRFQVPGEIEAEFAPIGAKSVLKKILTYRDPAPFYFPKGKGLEAIPDAPVALSSWLSEEELDYYANKFEQTGFTGAVNYYRALPINWELTAPWTGAQVKVPTKFIVGEFDLVYHIPGAKEYIHNGGFKKDVPLLEEVVVLEGAAHFVSQERPHEISKHIYDFIQKF

SEQ ID NO: 7 - protein, 398 aa, Aspergillus niger
  AnEH
MSAPFAKFPSSASISPNPFTVSIPDEQLDDLKTLVRLSKIAPPTYESLQADGRFGITSEWLTTMREKWLSEFDWRPFEARLNSFPQFTTEIEGLTIHFAALFSEREDAVPIALLHGWPGSFVEFYPILQLFREEYTPETLPFHLVVPSLPGYTFSSGPPLDKDFGLMDNARVVDQLMKDLGFGSGYIIQGGDIGSFVGRLLGVGFDACKAVHLNLCAMRAPPEGPSIESLSAAEKEGIARMEKFMTDGLAYAMEHSTRPSTIGHVLSSSPIALLAWIGEKYLQWVDKPLPSETILEMVSLYWLTESFPRAIHTYRETTPTASAPNGATMLQKELYIHKPFGFSFFPKDLCPVPRSWIATTGNLVFFRDHAEGGHFAALERPRELKTDLTAFVEQVWQK

SEQ ID NO: 8 - protein, 558 aa, Pseudomonas putida
  AlkJ
MYDYIIVGAGSAGCVLANRLSADPSKRVCLLEAGPRDTNPLIHMPLGIALLSNSKKLNWAFQTAPQQNLNGRSLFWPRGKTLGGSSSINAMVYIRGHEDDYHAWEQAAGRYWGWYRALELFKRLECNQRFDKSEHHGVDGELAVSDLKYINPLSKAFVQAGMEANINFNGDFNGEYQDGVGFYQVTQKNGQRWSSARAFLHGVLSRPNLDIITDAHASKILFEDRKAVGVSYIKKNMHHQVKTTSGGEVLLSLGAVGTPHLLMLSGVGAAAELKEHGVSLVHDLPEVGKNLQDHLDITLMCAANSREPIGVALSFIPRGVSGLFSYVFKREGFLTSNVAESGGFVKSSPDRDRPNLQFHFLPTYLKDHGRKIAGGYGYTLHICDLLPKSRGRIGLKSANPLQPPLIDPNYLSDHEDIKTMIAGIKIGRAILQAPSMAKHFKHEVVPGQAVKTDDEIIEDIRRRAETIYHPVGTCRMGKDPASVVDPCLKIRGLANIRVVDASIMPHLVAGNTNAPTIMIAENAAEIIMRNLDVEALEASAEFAREGAELELAMIAVCM

SEQ ID NO: 9 - protein, 534 aa, Sphingomonas sp. HXN-200
  Sp1814
MTQESDNSTFDYIIVGGGSGGCVVANRLSENPDISVCLIELGGDDDQTLVNVPLGMAAMLPTKINNYGFETVPQPGLNGRKGYQPRGRVLGGSSSINAMCYIRGQAEDYDDWAANGAPHWSYKDVLPYFRKSECNERGEDEFHGASGPLNVADHRSPNPVSDMFIEAASERQHRVNLDFNGAAQEGVGRYQVTQKDGRRWNVARGYLRPIMGRSNLTVMTRTKALRILMSGSRATGLEVLRHKKTQKLTAAEGVVLASGAFGTPHLLMLSGLGPEAELKRNGIPVLFNIPGVGQNLQDHPDHISVYRANSPDLFAISPGGVARIGASIPDYMRHGRGPLTSNAAEAGAFLSTKGRGNRPDVQMHFVHGIIDDHSRKIHFGGGMSCHVCVLRPESRGSVTLGSADPMAPPVIDPNFLVTDTDVATMLAGFKMVREIMESPALKSIRGAEMFTKGITKDAALIEALRSRTDTVYHPVGTCRMGTDEAAVCDPNLKVCGIDNIWIADASIMPTLVSGNTNAPVVMIAERASEFILNH

SEQ ID NO: 10 - protein, 375 aa, Equus caballus
  HlADH
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 11 - protein, 757 aa, Gluconobacter oxydans 621H
  GoADH
MTSGLLTPIKVTKKRLLSCAAALAFSAAVPVAFAQEDTGTAITSSDNGGHPGDWLSYGRSYSEQRYSPLDQINTENVGKLKLAWHYDLDTNRGQEGTPLIVNGVMYATTNWSKMKALDAATGKLLWSYDPKVPGNIADRGCCDTVSRGAAYWNGKVYFGTFDGRLIALDAKTGKLVWSVYTIPKEAQLGHQRSYTVDGAPRIAKGKVLIGNGGAEFGARGFVSAFDAETGKLDWRFFTVPNPENKPDGAASDDILMSKAYPTWGKNGAWKQQGGGGTVWDSLVYDPVTDLVYLGVGNGSPWNYKFRSEGKGDNLFLGSIVAINPDTGKYVWHFQETPMDEWDYTSVQQIMTLDMPVNGEMRHVIVHAPKNGFFYIIDAKTGKFITGKPYTYENWANGLDPVTGRPNYVPDALWTLTGKPWLGIPGELGGHNFAAMAYSPKTKLVYIPAQQIPLLYDGQKGGFKAYHDAWNLGLDMNKIGLFDDNDPEHVAAKKDFLKVLKGWTVAWDPEKMAPAFTINHKGPWNGGLLATAGNVIFQGLANGEFHAYDATNGNDLYSFPAQSAIIAPPVTYTANGKQYVAVEVGWGGIYPFLYGGVARTSGWTVNHSRVIAFSLDGKDSLPPKNELGFTPVKPVPTYDEARQKDGYFMYQTFCSACHGDNAISGGVLPDLRWSGAPRGRESFYKLVGRGALTAYGMDRFDTSMTPEQIEDIRNFIVKRANESYDDEVKARENSTGVPNDQFLNVPQSTADVPTADHP

SEQ ID NO: 12 - protein, 483 aa, Pseudomonas putida
  AlkH
MTIPISLAKLNSSADTHSALEVFNLQKVASSARRGKFGIAERIAALNLLKETIQRREPEIIAALAADFRKPASEVKLTEIFPVLQEINHAKRNLKDWMKPRRVRAALSVAGTRAGLRYEPKGVCLIIAPWNYPFNLSFGPLVSALAAGNSVVIKPSELTPHTATLIGSIVREAFSVDLVAVVEGDAAVSQELLALPFDHIFFTGSPRVGKLVMEAASKTLASVTLELGGKSPTIIGPTANLPKAARNIVWGKFSNNGQTCIAPDHVFVHRCIAQKFNEILVKEIVRVYGKDFAAQRRSADYCRIVNDQHFNRINKLLTDAKAKGAKILQGGQVDATERLVVPTVLSNVTAAMDINHEEIFGPLLPIIEYDDIDSVIKRVNDGDKPLALYVFSEDKQFVNNIVARTSSGSVGVNLSVVHFLHPNLPFGGVNNSGIGSAHGVYGFRAFSHEKPVLIDKFSITHWLFPPYTKKVKQLIGITVKYLS

SEQ ID NO: 13 - protein, 499 aa, Escherichia coli
  EcALDH
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLEPLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCGKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 14 - protein, 493 aa, Sphingomonas sp. HXN-200
  Sp2643
MASAPAVHLHLGHEQRTSGSGGTHPHLHPVKQVVQADIPLAGAKEVEEAVARAAAVQEDWRRTPPETRRDILNRLADLLEANKRTLAEMAALDGGTTLMVGERGVDTAVGWTRYYAGWCDKMSGELISTFDTRGELSYTVPEPIGIVGIIITWNGPLISLGMKVAAALAAGNCVICKPAEITPFAPEMFAQLCKQAGVPDGVLSILPGTAEAGEAIVRHKKIRKISFTGGPITARKILTACAEEIKPSVMELGGKSASLVFPDCDLQAAAERAVFWTVGCLSGQGCALPTRQLVHADVYDDFVARLKAIIGQFKVGDPMDPTVAVGPVINTAAVDRILGMFERAKADGAAKFELGGGRCGGELADGNFIEPTLIVDADPDHEISQVEIFGPAVVVMKFHTEDEAIAIANNSEYGLAAYIQSNDLQRVHRLSERLSAGGVYNNGGFQINPHTPFGGIGISGFGKEGGKAGIDEFLHYKTVTIGVGAPIFPKQEA

SEQ ID NO: 15 - protein, 484 aa, Sphingomonas sp. HXN-200
  Sp2860
MATAIKQDIAGETARMHEVLAAQKASFTAAMPESLAVRRDRIDRAIALLVDNAEEFAKAVSEDFGHRSRDQTLMTDIMPSVSALKHAKKHMAAWSKGEKRKPTFPLGLLGAKAEVVYQPKGVVGVVAPWNFPVGMVFVPMAGILAAGNRAMVKPSEFTENVSALMARLVPDYFDESEMAVFTGDADVGIAFSKLAFDHMIFTGATSVGRHIMRAAADNLVPVTLELGGKSPTFIGRSANKDLVGQRVALGKMMNAGQICLAPDYLLVAEDQEGAVIDSVTKGAAALYPTLLANDDYTSVVNTRNYDRLQSYLTDARDKGAEVIEVNPGGEDFASSNGHKMPLHIVRNPTDDMKVMQEEIFGPILPVKTYKSIDDAIDYVNANDRPLGLYYFGQDKSEEDRVLTRTISGGVTVNDVLFHNAMEDLPFGGVGPSGMGNYHGVDGFRTFSHARAVYRQPKLDVAGLAGFKPPYGKATAKTLAKELKK

SEQ ID NO: 16 - protein, 418 aa, Streptomyces coelicolor A3(2)
  AldO
MSDITVTNWAGNITYTAKELLRPHSLDALRALVADSARVRVLGSGHSFNEIAEPGDGGVLLSLAGLPSVVDVDTAARTVRVGGGVRYAELARVVHARGLALPNMASLPHISVAGSVATGTHGSGVGNGSLASVVREVELVTADGSTVVIARGDERFGGAVTSLGALGVVTSLTLDLEPAYEMEQHVFTELPLAGLDPATFETVMAAAYSVSLFTDWRAPGFRQVWLKRRTDRPLDGFPYAAPAAEKMHPVPGMPAVNCTEQFGVPGPWHERLPHFRAEFTPSSGAELQSEYLMPREHALAALHAMDAIRETLAPVLQTCEIRTVAADAQWLSPAYGRDTVAAHFTWVEDTAAVLPVVRRLEEALVPFAARPHWGKVFTVPAGELRALYPRLADFGALAGALDPAGKFTNAFVRGVLAG

SEQ ID NO: 17 - DNA, 32 bp, Artificial Sequence
  sythetic oligonucleotide
actgtcatgaaaaagcgtatcggtattgttgg

SEQ ID NO: 18 - DNA, 37 bp, Artificial Sequence
  sythetic oligonucleotide
actggaattctcatgctgcgatagttggtgcgaactg

SEQ ID NO: 19 - DNA, 30 bp, Artificial Sequence
  sythetic oligonucleotide
actgcatatgacgctgaaaaaagatatggc

SEQ ID NO: 20 - DNA, 33 bp, Artificial Sequence
  sythetic oligonucleotide
actgggtacctcaattcagtggcaacgggttgc

SEQ ID NO: 21 - DNA, 47 bp, Artificial Sequence
  sythetic oligonucleotide
actggaattctaaggagatttcaaatgacgctgaaaaaagatatggc

SEQ ID NO: 22 - DNA, 33 bp, Artificial Sequence
  sythetic oligonucleotide
atcgcatatgatgaacgtcgaacatatccgccc

SEQ ID NO: 23 - DNA, 35 bp, Artificial Sequence
  sythetic oligonucleotide
atcgctcgagtcaaagatccatctgtgcaaaggcc

SEQ ID NO: 24 - DNA, 50 bp, Artificial Sequence
  sythetic oligonucleotide
actgggtacctaaggagatatatcatgatgaacgtcgaacatatccgccc

SEQ ID NO: 25 - DNA, 31 bp, Artificial Sequence
  sythetic oligonucleotide
actgcatatggagaaaatcgaacacaagatg

SEQ ID NO: 26 - DNA, 31 bp, Artificial Sequence
  sythetic oligonucleotide
actgctcgagttagaatttttgaataaaatc

SEQ ID NO: 27 - DNA, 47 bp, Artificial Sequence
  sythetic oligonucleotide
actgggtacctaaggagatatatcatggagaaaatcgaacacaagat

SEQ ID NO: 28 - DNA, 34 bp, Artificial Sequence
  sythetic oligonucleotide
actgtcatgacgcaagagtcagataatagtactt

SEQ ID NO: 29 - DNA, 35 bp, Artificial Sequence
  sythetic oligonucleotide
actgagatctttaatggttcaagatgaattccgac

SEQ ID NO: 30 - DNA, 34 bp, Artificial Sequence
  sythetic oligonucleotide
cgcggatccatgtacgactatataatcgttggtg

SEQ ID NO: 31 - DNA, 33 bp, Artificial Sequence
  sythetic oligonucleotide
cgcgtcgacttacatgcagacagctatcatggc

SEQ ID NO: 32 - DNA, 32 bp, Artificial Sequence
  sythetic oligonucleotide
attccatatgaccataccaattagcctagcca

SEQ ID NO: 33 - DNA, 35 bp, Artificial Sequence
  sythetic oligonucleotide
ccgctcgagtcagctcaaatacttaactgtgatac

SEQ ID NO: 34 - DNA, 39 bp, Artificial Sequence
  sythetic oligonucleotide
actgggatccgatgtacgactatataatcgttggtgctg

SEQ ID NO: 35 - DNA, 35 bp, Artificial Sequence
  sythetic oligonucleotide
actgagatctttacatgcagacagctatcatggcc

SEQ ID NO: 36 - DNA, 49 bp, Artificial Sequence
  sythetic oligonucleotide
cgagatcttaaggagatatataatgacagagccgcatgtagcagtatta

SEQ ID NO: 37 - DNA, 36 bp, Artificial Sequence
  sythetic oligonucleotide
actgctcgagttaataccgtacacacaccgacttag